(19) United States
(12) Patent Application Publication
(10) Pub. No.: US 2026/0102509 A1
(43) Pub. Date: Apr. 16, 2026

(54) METAL OXIDE BIOLOGICAL IONOMER AND METHODS

(71) Applicant: Peter Butzloff, Walnut, CA (US)

(72) Inventor: Peter Butzloff, Walnut, CA (US)

(21) Appl. No.: 18/913,941

(22) Filed: Oct. 11, 2024

(51) Int. Cl. A61K 47/69; A61K 9/51; A61K 31/00; A61K 31/616; A61K 41/00

(52) U.S. Cl. CPC A61K 47/6929; A61K 9/5115; A61K 31/616; A61K 31/658; A61K 41/0047

(57) ABSTRACT

A nanoparticle compound comprising buckminsterfullerene C60 bonded to ester functional groups of pyruvic, citric, and hyaluronic acids, with oxygen-bridged components selected from vanadium, gallium, and bismuth is provided. At least one component enables quantum-mechanical spin-wave activation. Ultrasonic irradiation activates the compound, inducing electrical induction for tissue healing and cell growth. The compound integrates with native and 3D-printed tissues, enhancing their properties through interpenetrating networks. It exhibits anti-inflammatory, antioxidant, and antibacterial effects, reinforces connective tissue, and modulates stem cell growth. The compound treats arthritis, enhances gut health and barrier function, supports bone health, improves voice quality, counters cancer cell proliferation, and mitigates osteoporosis. The gallium component creates an alkaline environment for calcium deposition, while vanadium enhances chondrogenic differentiation. Administration methods include topical application, oral ingestion, injection, and infusion through bandages or sutures. The compound serves as an adjuvant for analgesics, enhancing their delivery and efficacy.

### CROSS-REFERENCE TO RELATED APPLICATIONS

This application is a continuation-in-part of U.S. provisional application 63/590,449 filed on Oct. 15, 2023 and entitled “METAL OXIDE BIOLOGICAL IONOMER AND METHODS” which is incorporated by reference herein in its entirety.

### BACKGROUND OF THE INVENTION

### Field of the Invention

This invention is a composition of buckminsterfullerene citric metal pyruvate hyaluronate, wherein the metal oxides are covalently bonded within a biopolymer forming a self-healing biological ionomer; hereinafter this composition is termed the nanoparticle compound. Methods of irradiation are provided to generate non-contact electrical induction into living tissues to speed healing and stimulate stem cell division. Induced electromagnetic oscillations generated with the nanoparticle compound provide a quantum mechanical based medicine and versatile drug delivery targeting vehicle with an accelerated release rate that can be enabled by quantum spin-waves. It effectively treats conditions such as arthritis, restores bone density, inhibits cancer cell growth, offers neuroprotection, and serves as a broad-spectrum antimicrobial agent. The electrostatic ionomeric self-healing properties of the nanoparticle compound are provided as it integrates with biological connective tissue.

The nanoparticle compound suppresses reactive oxygen species and expedites molecular reinforcement of both endogenous and implanted stem cells, including the mitigation of inflammation associated with induced pluripotent stem cell (iPSC) implantation or directed growth. Through chemical bonding, metal ions interact with various components, with a soft outer shell facilitating integration and interpenetration with biopolymers like collagen, chondroitin sulfate, proteoglycans, fatty acids, proteins, and hyaluronic acid, thereby enhancing properties such as elastic modulus, wear resistance, and compressibility of living tissues. Applications encompass anti-inflammation and tissue reinforcement, including molecular sutures in vascular surgery, cartilage reinforcement in joints, healing burn injuries, and restoring nerves, neurons, tight junctions, and damage resulting from traumatic brain injuries (TBI). The nanoparticle compound also aids in glycolysis regulation for cancer treatment. Delivery methods include oral ingestion, topical application, injection, implantation, and infusion through bandages or via implanted sutures or surgical threads.

### Background Art

The use of hollow carbons to create stable pores in liquids relies on charged reactive sites called ionomers to create molecular scale interactions capable of stabilizing the pore structures in liquid compositions. The presence of migrating pores is critical to biopolymer remodeling. For example, a negatively charged molecule can become part of the structure of an ionomer by adding positive counter-charged molecules. The interaction between unlike charges separated in space but abutting can provide lubricious flowing liquid porosity. To achieve a self-healing effect, the charge attractions must form a metastable solid where the volume of the pores is stabilized by the formation of oriented counter-charges.

Biological tissues utilize processes for protein remodeling, such as in the Krebs Cycle, also known as the citric acid cycle of respiration. Failure of catabolism exhibits in the build-up of pathogenic misfolded proteins within the organelles or compartments within living cells.

Catabolic and anabolic cycles become impaired with age or disease states as impairments to the chemistry needed to repair and replenish the molecular morphology, pores, and microstructures of living connective tissues. Failure to recycle oxidized peptides and proteins and are all considered aspects of insufficiency of remodeling of connective soft tissues. This can result in diseases such as arthritis. All biological substances in the human body, including bone, have connective molecular elements. The failure to remodel and repair these soft elements is a fundamental problem of age related disease where improved treatment methods are sought, for example to replace deterioration of the cartilaginous tissues.

As the global average age has increased, the costs of arthritis have increased as well, so that presently 1 in 6 people suffer from this condition. Of these patients, there is a significant economic cost for the provision of pain management drugs such as methotrexate, where many people continue to suffer because of a suboptimum response to such drugs as the human body responds to such treatments in ways that are increasingly ineffective over time. There are no cures for arthritis at present because no sufficiently fundamental advance has been made to understand arthritis in the context of tissue remodeling.

At present, arthritis is treated with nonsteroidal anti-inflammatory drugs (NSAIDs), glucocorticoid joint injections, or in the most severe cases, to prescribe prednisone, an extremely dangerous drug that has severe side effects, can cause kidney failure, and may cause death if dosages are missed for the control of inflammatory symptoms. While every effort is made to avoid narcotic analgesic dependency, eventually drug dependency cannot be avoided because the source of the pain of arthritis is never appropriately addressed. A practical alternative connective tissue repair molecule is sought to remediate catabolic dysfunction in the remodeling of cartilage. Such remediation must operate on synovial fluids as well as the compressibility of cartilage to compensate for the effects of aging.

Arthritis, osteoporosis, neoplastic disease (cancers, tumors), share dysfunctions associated with the remodeling of connective tissues. Billions of dollars of health care costs to all societies can be saved for aging populations if multifunctional enhancements can be combined to improve the remodeling of biological connective tissues as a non-toxic and biocompatible supplement or drug.

Buckminsterfullerene derivatives such as Fullerenol have demonstrated dose dependent efficacy against various antibiotic-resistant microbial pathogen strains, including MRSA (methicillin-resistant Staphylococcus aureus) and E. coli by disruption of bacterial membrane disruption, reduced biofilm formation, and interference with bacterial signaling pathways. For example, fullerenol-gentamicin conjugates appear to have lower toxicity to human cells compared to traditional antibiotics that may be used to combat inflammation, infection, and to reduce the innate immune responses associated with inflammation without unfavorable cytotoxic effects towards eukaryotic cells. Research is ongoing into effective delivery methods for fullerene derivatives, including nano formulations that can enhance their stability and bioavailability to improve their practical application in preventing or treating infections such as those that may associated with arthritis, gut dysbiosis, and some types of inflammation such as those associated with autoimmune diseases.

Various metal oxides have shown useful medicinal properties in prior-art applications. Bismuth oxides such as bismuth subsalicylate, the active ingredient in Pepto-Bismol, have been demonstrated to be useful in treating diarrhea, and inflammation, especially for gastritis because it has antimicrobial properties against various pathogens, including Helicobacter pylori, which is associated with peptic ulcers. Vanadium compounds, including vanadyl sulfate, demonstrate insulin-mimetic effects, enhancing glucose regulation, while also possessing anticancer properties by inhibiting cell proliferation and promoting apoptosis. Vanadium oxide (VO₂) compounds also are reported to show promise in improving cardiovascular health, and in favoring cartilage growth in vitro, underscoring their clinical versatility in addressing various health concerns, however their efficacy, delivery, and better targeting to tissues is capable of significant improvement before commercial viability can be sufficiently realized. Gallium metal hydroxides are generally known for their inability to form free radicals yet mimic the biochemistry of iron under physiological conditions in both the +2 and the +3 oxidation states. Gallium metal is reported to favor osteoclast stem cell growth, however, the reported mild toxicity of gallium hydroxide on sudden release requires reconsideration of the pharmacokinetics before it can become safely commercialized. The low toxicity and similar oxidation states of gallium, vanadium, and bismuth oxides and hydroxides make these metal cations attractive for the design of new therapeutic substances by creating derivatives from them.

An objective of the present invention is to provide a composition of matter (the nanoparticle compound) to help remodel biological connective tissues in a health promoting and strength reinforcing manner by the introduction of positive charged bacteriostatic metal cations capable of flexibly reconfiguring hyaluronic acid among fatty acid, C60, and amine type ionomer linking elements.

### SUMMARY OF THE INVENTION

The present invention relates to a nanoparticle compound and methods for its use in various medical applications. The compound comprises a buckminsterfullerene C60 core bonded to ester functional groups of pyruvic acid, citric acid, and hyaluronic acid, along with oxygen-bridged components selected from vanadium, gallium, and bismuth. This unique structure enables quantum-mechanical spin-wave activation, allowing for non-invasive stimulation through ultrasonic irradiation.

The nanoparticle compound demonstrates versatile applications in treating arthritis, enhancing gut health, improving barrier function, supporting bone health, and enhancing voice quality. It exhibits anti-inflammatory, antioxidant, and antibacterial properties, while also showing promise in cancer treatment by inhibiting glycolysis in cancer cells.

The compound's ability to form interpenetrating networks with native tissues and 3D-printed scaffolds enhances its efficacy in tissue engineering and regenerative medicine. Its unique properties allow for controlled drug delivery, stem cell stimulation, and acceleration of healing processes.

Methods of activating and delivering the compound involve ultrasonic irradiation, which induces electrical induction within the nanoparticle structure. This activation can be modulated to control delivery rates and stimulate targeted cellular responses.

The invention provides a novel approach to addressing various medical conditions through a single, multifunctional nanoparticle compound, offering potential advancements in personalized medicine and targeted therapies.

These and other advantages of the present invention will be further understood and appreciated by those skilled in the art by reference to the following written specification, claims, and appended drawings.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

In one aspect, the nanoparticle compound, termed as such, is a nano-surfactant designed for enhanced healing and molecular reinforcement. Comprising esters of pyruvic, citric, and hyaluronic acids with oxygen-bridged vanadium, gallium, and bismuth, it functions as a biologically compatible ionomer.

In a related aspect, non-contact ultrasonic irradiation induces electrical induction within the nanoparticle compound emplaced on or injected into a gap or discontinuous region in living tissues. This energy electrically activates biochemical bonding and crosslinking of the nanoparticle compound in that region to provide a scaffold for the purpose of cell adhesion to bridge that gap or discontinuous region, as the electric current is provided through electromagnetic voltages induced within the vanadium II oxide component and conducted into and through the fullerene carbon nanoparticles of that composition.

In a related aspect, non-contact ultrasonic irradiation induces electrical induction within the nanoparticle compound integrated into living tissues. This energy accelerates healing and stimulates stem cell division through electromagnetic oscillations within the vanadium II oxide component.

In another related aspect, directed ultrasound modulates substance delivery rate by the nanoparticle compound. Increased ultrasonic energy yields accelerated substance release.

A related aspect involves quantum-mechanical activation of cell growth through quantum spin-waves, enabling electric charge manipulation by the vanadium dioxide group.

In another related aspect, the nanoparticle compound serves as an adjuvant for delivering analgesics like aspirin or cannabidiol.

In another aspect, the nanoparticle compound treats all forms of arthritis.

In a related aspect, the nanoparticle compound is incorporated into native tissues, including collagen, chondroitin sulfate, proteoglycans, fatty acids, and proteins, enhancing their properties by forming counterionic peptide linkages to interpenetrate and reinforce the somatic biopolymers, improving their flexibility, elastic modulus, wear resistance, and compressibility.

In a related aspect, the nanoparticle compound is introduced into 3D-printed tissues with similar shape and composition as native organs, promoting integration with seeded replacement cells and compatible substances.

In another aspect, the nanoparticle compound provides anti-inflammation and connective tissue reinforcement, accelerating restorative homeostasis and stem cell growth post-injury or surgery.

In a related aspect, the nanoparticle compound modulates stem cell growth, facilitating integration of various stem cells within endogenous tissue or biocompatible tissue scaffolds.

In another aspect, the nanoparticle compound controls gut inflammation, including dysbiosis, for resolving diarrhea or sepsis.

In a related aspect, the nanoparticle compound alters gut microflora, promoting healthy bacteria and enhancing nutrient digestion.

In a related aspect, the nanoparticle compound reinforces barrier functions in the blood-brain-barrier and the gut lining, resolving conditions like ulcerative colitis associated with leaky barriers.

In another aspect, the nanoparticle compound offers neuroprotection, addressing neuritis, and inflammation of the nervous system, while exhibiting bacteriostatic, anti-inflammatory, and antioxidant functions.

In another aspect, the nanoparticle compound is used for voice enhancement, and improves the flexibility of the larynx, enhancing vocal quality and control.

In another aspect, the nanoparticle compound serves as a treatment for arthritis, with diverse administration methods including topical application, oral solutions, powders, sub-lingual patches, injection, implantation, or infusion by bandages.

In another aspect, the nanoparticle compound forms nanomolecular sutures for tissue healing, microvascular repair, and embolism prevention.

In another aspect, the nanoparticle compound counters cancer cell proliferation and neoplasms by reducing glycolysis and binding with phosphofructokinase, inhibiting its operation.

In a related aspect, the nanoparticle compound catalyzes the recombination and quenching of free radicals, reducing radiation and chemotherapy-induced damage to healthy tissues.

In another aspect, the nanoparticle compound exchanges gallium ions with Fe3+, encouraging the deposition of ionic calcium to mitigate and reverse osteoporosis.

In another aspect, the introduction of nanoparticle compound involves infusion into threads, fibrous patches, transdermal patches, or sublingual dissolution patches.

In a related aspect, the nanoparticle compound administration can be by oral ingestion, topical application, or infusion by a bandage or emplaced sutures abutting cut or damaged tissues.

### BRIEF DESCRIPTION OF THE DRAWINGS

Preferred embodiments of the invention will now be described, by way of example, with reference to the accompanying drawings, in which:

FIG. 1 illustrates polyhydroxylated buckminsterfullerene reaction with sodium pyruvate, forming an intermediate part of the nanoparticle compound composition.

FIG. 2 illustrates buckminsterfullerene pyruvic ester reaction with metal cations to include gallium, forming an intermediate part of the nanoparticle compound composition.

FIG. 3 illustrates citric with metal cations to include gallium, forming an intermediate part of the nanoparticle compound composition.

FIG. 4 illustrates hyaluronic acid biopolymer with metal cations to include gallium, forming an intermediate part of the nanoparticle compound composition.

FIG. 5 illustrates the vanadium II oxide (VO₂) nanoparticle reaction with gallium.

FIG. 6 illustrates the metal oxide conjugated citric hyaluronic pyruvate esters of buckminsterfullerene designating the nanoparticle compound molecular structure of the present invention.

FIG. 7 is a schematic illustration of the drug delivery and incorporation of the nanoparticle compound to intercalate and reinforce connective tissues including those of implanted stem cells.

FIG. 8 illustrates the use of the nanoparticle compound to mediate calcium bonding for bone density recovery from osteoarthritis, osteonecrosis, and conditions involving osteoporosis.

FIG. 9 illustrates the use of the nanoparticle compound to treat hypoxic tumor and cancer cells enabling apoptosis or cell death of proliferative neoplasms.

FIG. 10 is a flowchart showing a method of synthesizing the nanoparticle compound.

FIG. 11 is a flowchart showing alternative formulations to deliver the nanoparticle compound.

FIG. 12 illustrates the use of the nanoparticle compound to mediate anti-inflammation, swelling reduction, and bone alignment recovery on swelling reduction from arthritis.

FIG. 13 illustrates larynx treatment methods for vocalists by means of the nanoparticle compound of the present invention.

FIG. 14 illustrates the personal administration of topical nanoparticle compound skin formulation.

FIG. 15 illustrates the method of non-contact activation of the nanoparticle compound inside of a targeted internal organ by the external application of directed energy.

FIG. 16 illustrates experimental FTIR data for sodium saponified poly-oxylated-buckminsterfullerene.

FIG. 17 illustrates experimental FTIR data for hyaluronic acid.

FIG. 18 illustrates experimental FTIR data for pyruvic acid.

FIG. 19 illustrates experimental FTIR data for the pyruvate ester of buckminsterfullerene.

FIG. 20 illustrates experimental FTIR data for the hyaluronic pyruvate esters of buckminsterfullerene.

FIG. 21 illustrates experimental FTIR data for the metal oxide conjugated citric hyaluronic pyruvate esters of buckminsterfullerene as the nanoparticle compound of the present invention.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### DETAILED DESCRIPTION OF THE INVENTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

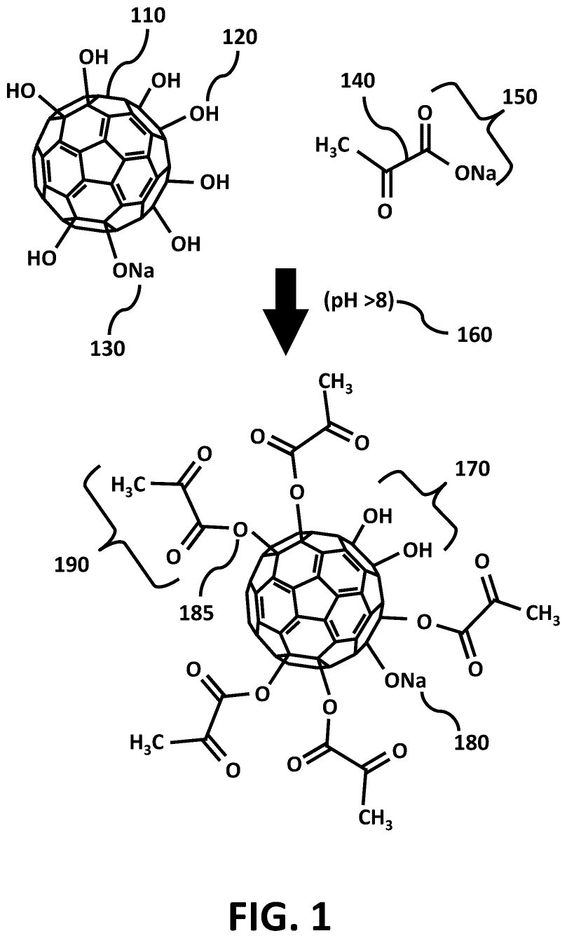

FIG. 1 illustrates molecular structures for an esterification reaction between polyhydroxylated buckminsterfullerene and sodium pyruvate. Polyhydroxylated buckminsterfullerene 110, also known as fullerenol, is a commercially available, water-dispersible nanoparticle with at least 6 to 22 hydroxyl groups, nominally sixteen hydroxyl groups 120. Either fullerenol or saponified fullerenol 130 is suitable for synthesizing the nanoparticle compound of the present invention. Sodium pyruvate 140 includes a saponified carboxylic acid (COONa) functional group 150. The reaction involves shear mixing saponified fullerenol with sodium pyruvate in the presence of sodium hydroxide at a pH greater than 8, as indicated at 160. Dry sodium hydroxide is added to initiate the reaction and achieve the required elevated pH. The reaction proceeds in the direction shown by the heavy solid black arrow pointing downward, yielding an intermediate buckminsterfullerene pyruvate ester molecule. In this intermediate, most of the hydroxylated groups 170 or saponified fullerenol groups 180 have been esterified, as represented by the bridging oxygen 185 of at least about five representative pyruvate functional groups 190.

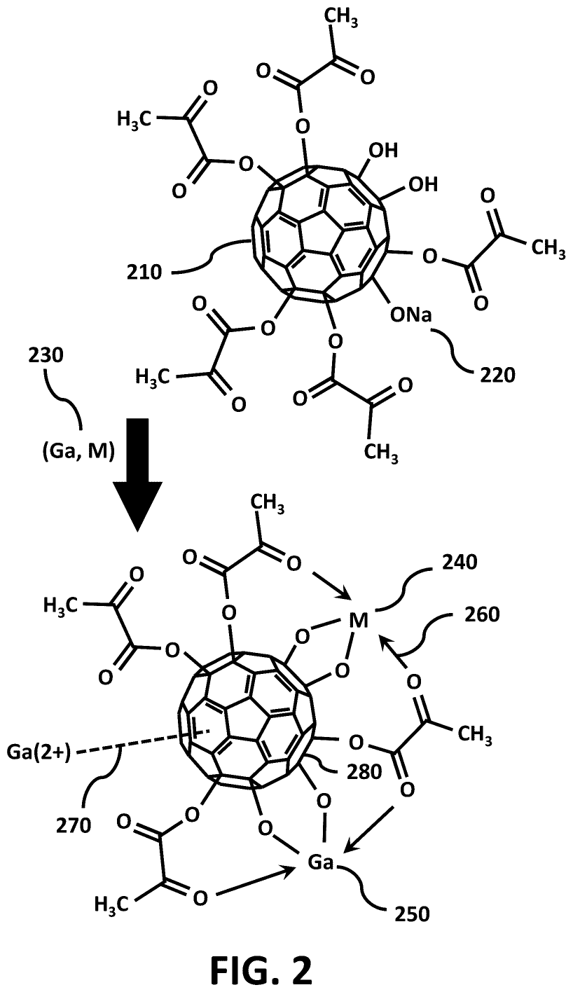

FIG. 2 schematically illustrates a metal cation reaction with buckminsterfullerene hydroxyl pyruvate ester. A partly saponified buckminsterfullerene hydroxyl pyruvate ester 210 is reactively shear mixed with other reactants. Some groups of the reactant 210 may be saponified 220. The other reactants include a multiplicity of gallium (Ga) metal, or another multivalent transition metal selected from bismuth (Bi) or vanadium (V) in any combination, represented by the letter M, 240. Bismuth, gallium, vanadium and their oxides, being weakly diamagnetic, contribute bacteriostatic properties to the reaction. Nanoparticles of Vanadium II oxide (VO2) comprise the greater multiplicity of M, 240 in this chemical reaction to provide significant quantum mechanical spin-wave oscillations via the inductive magneto-electric Faraday effect. M reacts to displace hydrogen from available hydroxyl (OH) groups or saponified hydroxyl groups (ONa) to create a bridging metal cation M, 240 or a bridging gallium cation Ga, 250. An electron pair from any proximal carbonyl oxygen may form a weak bond with the metal oxide cation, indicated by the multiplicity of thin black arrows in the bonded structure 260, arising from at least one proximal carbonyl oxygen atom. Cation M of an oxidation state of one or greater can form an aromatic pi-cation bond, 270, as shown for gallium of +2 oxidation state bonded with the carbon surface of the buckminsterfullerene functional group 280. The resulting product material serves as a reactive intermediate substance for the synthesis of the nanoparticle compound.

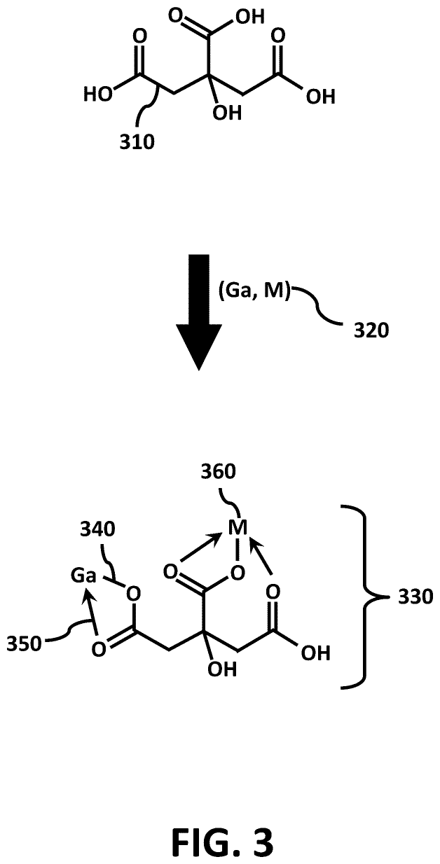

FIG. 3 illustrates a metal cation reaction with citric acid, producing reactive intermediates for the nanoparticle compound molecule. Citric acid, 310, is a tri-basic keto acid, and is a central enzyme in the Krebs Cycle (also known as the citric acid cycle) in living cells. High cytosolic citrate concentrations can inhibit neoplasms in cancer by interfering with phosphofructokinase, a rate-limiting catalyst in glycolysis. At physiological pH, one or two carboxyl groups (COOH) of citric acid may ionize. In aqueous solutions, the ionized hydrogen of the carboxylic acid functional group dissociates, leaving negatively charged oxygen atoms (COO—) that can chelate with metal cations of gallium, vanadium, and bismuth to form corresponding metal oxide citrates. The gallium cation (Ga) and citrate exhibit significant synergy in their anticancer properties. Vanadium (V) and bismuth (Bi) or their oxides, in combination with gallium (Ga), represented by M, 320, confer bacteriostatic properties. This reaction produces a metal oxide citrate complex 330 including gallium oxide 340. A lone pair of electrons on a proximal carboxylic oxygen atom partially shares with the gallium, as indicated by the small arrow 350. Gallium bonding 340 can provide an oxidation state of 1, and this reactive intermediate can further oxidize to higher gallium oxidation states of 2+ and 3+. Any indicated M, 360 provides antimicrobial function. Gallium must be present to confer bone densification (anti-porosity) properties, while vanadium is necessary for quantum spin wave properties when the metal oxide citrate intermediate product 330 is incorporated into the nanoparticle compound molecular structure.

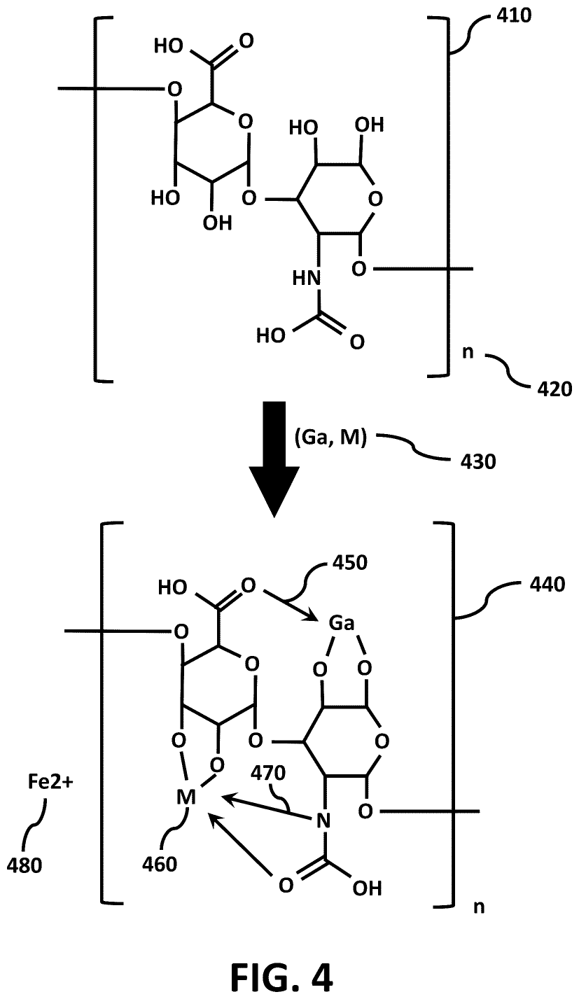

FIG. 4 illustrates a metal cations reaction with hyaluronic acid (HA). The bracketed structure 410 represents the repeating unit of the HA molecule. The HA has a minimum molecular weight of about 1 kilodalton, with nominally n=four repeating units (n), 420. The reaction involves reactive shear mixing of HA with 0.01 to about 10 percent by weight of gallium (Ga) or another multivalent transition metal selected from bismuth or vanadium in any combination, represented by M, 430, at a temperature greater than 30° C. The resulting hyaluronic metal oxide 440 contains covalently bonded metal cations. These cations attract the lone pair of electrons on proximal oxygen atoms, which are partly shared in the electrostatic bonding structure, as indicated by the small arrow 450. HA's nitrogen atoms can also share their lone pair of electrons with proximal metal cations 460, as indicated by the small arrow 470.

Metal cation M, 460 represents one of the bacteriostatic cations selected from bismuth, gallium, and vanadium. The ratio of these cations is adjustable based on the tissue type, location, and method of use. For example, substantial amounts of vanadium are desirable when utilizing the Faraday effect to induce dynamic conductivity changes in the VO2 component. Conversely, vanadium content can be minimized when satisfactory arthritis and bone densification treatment is obtainable from the gallium oxide component. Bismuth must be present in greater quantity than gallium for satisfactory treatment of diarrhea or sepsis to remediate pathogenic intestinal bacteria and restore gut health. An important function of metal atom M is to act as a cation exchange ion to extract iron cations from the site of inflammation and replace it by M while sequestering iron to the site where M was bonded, 460. Iron as free Fe2+, 480, is a major source of reactive oxygen species and free radicals, where this chemistry is known as the Fenton reaction. Iron cations are released by the human body as part the innate immune signaling response associated with a cascade of released cytokines and chemokines at the location of tissue damage or infection. However similar the chemistry of gallium is to iron, gallium is intrinsically unable to form free radicals with oxygen, thereby terminating this source of the inflammatory response at the location of damaged tissues. The proximity of the lone pair of electrons from the nitrogen atoms of hyaluronic acid, 470, will help to mask the captured iron at M, 460 from being released to form reactive oxygen species and cause inflammation at the site of damaged tissues in the gut or at the site of damaged cartilage tissues associated with various forms of arthritis.

The illustrated reaction product 440 is considered a reactive intermediate due to its limited ionomeric properties. Further processing to attach a C60 functional group is required to seal larger breaches at tight binding junctions of the intestine, induce a greater healing rate, and provide more robust molecular reinforcement of damaged connective tissues. These improvements are necessary to achieve the intended functionality of the present invention.

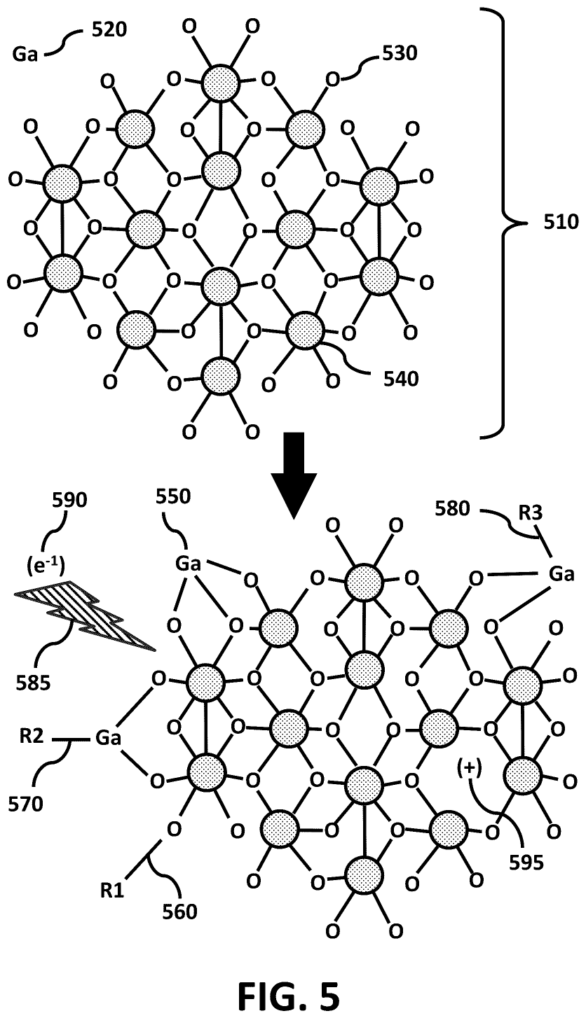

FIG. 5 illustrates a vanadium II oxide (VO2) nanoparticle reaction with gallium metal. A VO2 nanoparticle crystal 510 contains multiple oxygen atoms, represented by the atomic symbol O, 520. Gallium metal 520 reacts with oxygen 530 at the exposed surface of the VO2 nanoparticle, forming a gallium-coated VO2 nanoparticle structure (GaOxVO2), where x can vary from 1 to 3 oxygen bridges, as illustrated in the lower half of the figure. Gallium can form oxygen bridge bonds to three oxygen atoms, 550, or to different functional groups R1, R2, R3 (560, 570, 580 respectively) within the esterified structure of the nanoparticle compound.

VO2 undergoes a Mott-Hubbard spin-wave transition from a low-temperature insulating phase to a high-temperature metallic phase, accompanied by a structural transition from monoclinic M1 (space group P21/c) to rutile tetragonal R (space group P42/mnm), 510. Recent research confirms that the reversible phase change in VO2 is not the origin of the quantum mechanical spin waves responsible for the metal to insulator transition (MIT), also known as the metal to insulator change (MIC) or Faraday effect.

The conductivity change with temperature is notable: at room temperature, VO2 is electrically insulating, becoming electrically conducting above the spin wave transition temperature (Tc) of 68° C. for pure bulk VO2. Certain transition element metals and their oxides can significantly lower the Tc, allowing increased conductivity to be observed at or near room temperature (25° C.). In this invention, gallium (Ga) is used to partially achieve this Tc depression. While less effective than some transition elements in reducing Tc, gallium provides an inexpensive, non-toxic, bacteriostatic alternative for depressing the electrical conduction temperature Tc and enabling charge migration in GaOxVO2 for targeted irradiation-induced drug release in the human body.

Mild infrared, radio frequency, or acoustic energy irradiation can transiently induce Tc within the GaOxVO2 nanoparticle structure through joule heating without damaging delicate tissues. Ultrasonic irradiation of VO2 nanoparticles initiates spin-wave oscillations, providing sufficient penetration for non-contact, quantum mechanical-based electrical induction into living tissues and cells. This irradiation dynamically generates locations of voltage conduction 585 as some nanoparticles reach Tc at various times and locations. As phonon energy passes, some VO2 nanoparticles return to electrical insulation, expressing induced electrostatic charges where previously mobile electrons 590 or oxygen vacancies 595 become immobilized.

These changes in magnetic field, electric field, and electrostatic charged regions provide signals for irradiated adult stem cells and induced pluripotent stem cells to differentiate, contributing to non-contact accelerated healing. This non-contact irradiation method is preferable to direct voltage application in vivo, particularly in sensitive locations such as bone interiors, joint clefts, bone-cartilage transition zones, kidney nephrons, scarred liver or pancreas regions, or severe burn areas.

To maximize effectiveness, VO2 nanoparticles with diameters equal to or less than 20 nanometers are specified for the nanoparticle compound composition. The 10 nm VO₂ particles provided by American Elements Corporation, 10884 Weyburn Ave, Los Angeles, CA 90024 USA are suitable for achieving the desired MIT behavior. This size achieves conductivity near the percolation threshold, up to 8 times greater than bulk VO₂ properties (emerging for particles larger than 32 nanometers). An ideal 20-nanometer nanoparticle sphere contains approximately 502,000 vanadium (II) cations. Sufficient packing density of these nanoparticles, especially upon ultrasonic activation, is necessary to achieve the described electrical percolation between and among them when used in accordance with the present invention. Note: The nanoparticles in this illustration are not drawn to scale and represent much larger regions.

The use of ultrasound is one way to mechanically energize the VO₂ component of the nanoparticle compound to enable electric fields and currents to arise. Such currents are not piezoelectric in mechanism; instead, they arise from quantum-mechanical spin-wave activation. These voltages are sufficient to activate cell growth above the critical temperature of the MIT. The spin-waves, induced by pressure changes and mechanical activation by ultrasonic irradiation, create oscillating electric fields that can manipulate charges at the cellular level, stimulating growth and differentiation.

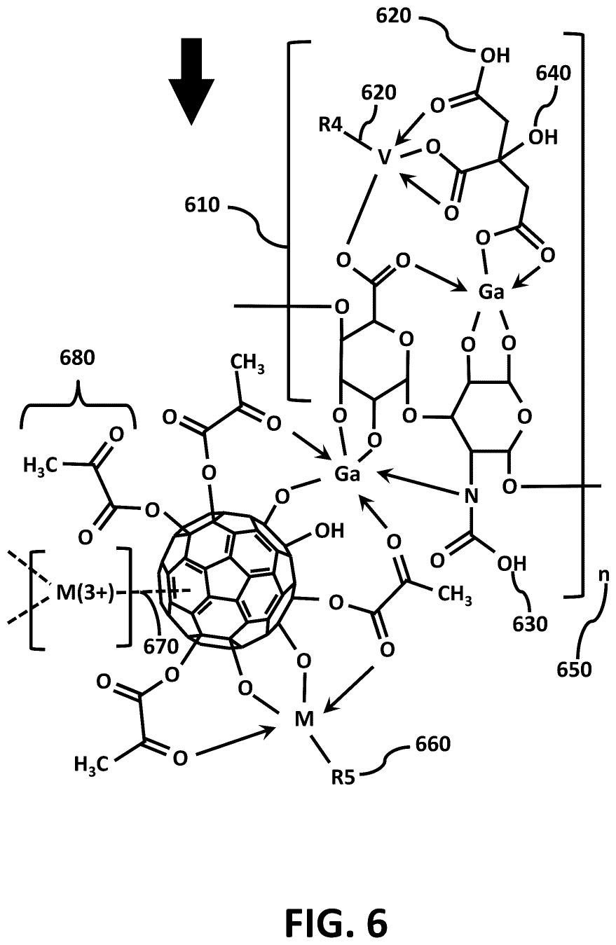

FIG. 6 illustrates the formation of a nanoparticle compound, comprising poly-esterified buckminsterfullerene pyruvate hyaluronic citric metal oxide. The figure shows a single representative polymeric element of hyaluronic acid 610 within square brackets, featuring multiple hydroxyl groups as part of carboxylic acids 620, 630. The presence of hyaluronic acid has the explicit purpose of integrating the nanoparticle compound with the typical biopolymers such as collagen that are used in 3D printed organ scaffolds by means of gallium oxygen and vanadium oxygen bridges. The molecular weight and thus the length of the hyaluronic acid molecule component determines the stiffness of the incorporated structures, which depends on the tissue type. Bone tissues have high modulus of elasticity and require greater gallium percentages to adjust the nanoparticle compound to favor osteogenesis whereas nephron structures in a kidney require significantly lower modulus of elasticity and require more vanadium percentage to adjust the nanoparticle compound; the identity of the nanoparticle compound constituents comprising this invention remain the same in all cases with the understanding that adjustments are necessary for proper organ function in each tissue type.

Gallium reduces glycolysis in cancer cells by inhibiting phosphofructokinase (PFK), a key regulatory enzyme in the pathway. Cancer cells rely on enhanced glycolysis for energy and biosynthesis to support rapid growth. By targeting PFK, gallium disrupts this metabolic dependency, impairing cancer cell proliferation and increasing their vulnerability to treatments. Additionally, gallium's interference with iron metabolism further hinders cellular processes critical for energy production and DNA synthesis. This dual action on glycolysis and iron metabolism highlights gallium's potential as a targeted cancer therapy, exploiting the metabolic weaknesses of cancer cells.

Gallium's medical applications leverage its chemical similarity to iron, particularly in its trivalent form (Ga3+), which follows biological pathways similar to iron (Fe3+) due to comparable ionic radius, electric charge, and ligand coordination. However, unlike iron, gallium cannot revert from Ga3+ to Ga2+, leading to its rapid excretion (69% in 24 hours, 91% in 48 hours). Gallium's uptake impairs iron-dependent ribonucleotide reductase, crucial for DNA synthesis and mitochondrial function, generating reactive species and triggering apoptosis through cytochrome C release and caspase-3 activation. Gallium nitrate exhibits anti-inflammatory and antimicrobial properties, effective against lupus, arthritis, fungi, bacteria, and even SARS-COV-2. In osteoporosis treatment, radioactive 67-gallium aids in bone metabolism by stabilizing the interface between collagen and calcium phosphate. Despite its effectiveness, gallium nitrate requires inconvenient continuous intravenous administration. Gallium compounds also show promise in cancer therapy; they localize in tumors similarly to iron, disrupting DNA replication by depriving ribonucleotide reductase of iron. This mechanism underpins gallium nitrate's use in treating non-Hodgkin's lymphoma and bladder cancer. Gallium maltolate has shown superior efficacy against liver carcinoma compared to gallium nitrate. The medical community anticipates further development of gallium compounds for clinical testing. Additionally, it has been reported that gallium quantum dots have been explored using ultrasonic actuation in water solutions but failed to form covalent bonds with C60. For this reason, the nanoparticle compound of present invention requires the use of linker molecules between C60 and gallium.

A vanadyl group 640 represents oxygen bridge formation due to vanadium dioxide presence, bonding to various components and creating metal oxide ligands or oxygen bridges. The antimicrobial activity of vanadium compounds extends to both bacteria and fungi, making them promising candidates for new treatments. Additionally, vanadium compounds support bone health by promoting formation and mineralization and offer neuroprotective benefits that may aid in managing neurodegenerative disorders. Their anti-inflammatory properties further contribute to their therapeutic value.

The hyaluronic acid molecule in this structure consists of approximately 4 repeating units, each comprising glucuronic acid and N-acetylglucosamine, with 407 atomic mass units, represented as ‘n’, 650. Hyaluronic acid molecular weight ranges from 407 to 40,700 Daltons, with ‘n=10’ typically forming the most robust nanoparticle compound for various medical applications. Chain length varies based on viscosity and tissue penetration requirements. Self-esterification at R5, 660 can extend the structure from metal atom M where M can be Vanadium, Bismuth, or Gallium having free radical quenching properties extending from the proximal C60 fullerene. The free radical quenching through the metal cation M is especially enabled by the pi-cation bonds between M being one of gallium, vanadium, or bismuth and the C60 group (indicated by dashed line at 670); free radical quenching is a well-documented antioxidant function of the C60 group which provides anti-inflammation properties; the chemistry of the metal cation M provides well-known antibacterial properties; the confluence of both M and C60 together enables both of these functions to provide both antibacterial and antioxidant mechanisms for the maintenance of homeostasis to cells and tissues treated with the nanoparticle compound. The ion exchange of M with metabolic free iron at the C60 functional group under physiological conditions is required to seal damaged tissue breaches at tight binding junctions of the intestine, induce the formation of an interpenetrating ionomer network between cartilage and the nanoparticle compound, and provide a robust molecular reinforcement of damaged connective tissues. These improvements are necessary to achieve the intended functionality of the present invention.

The nanoparticle compound structure exhibits high citrate concentrations in cell cytosol, which inhibit cancer neoplasms by interfering with glycolysis. Flexible and rotatable citrate esters form hemi-ortho bonding interactions via pi-carbonyl stacking, creating strong charge-transfer interactions. The illustrated structure represents one possible geometry, with various configurations possible, wherein oxygen bridges are utilized as covalent chemical bonding structures to link with biological molecules as functional groups essential to gut health to include hyaluronic acid, citric acid, and pyruvic acid, wherein the lipophilic delivery is improved by the selection of buckminsterfullerene hydroxyl. The nanoparticle compound's interaction with gut microflora promotes beneficial bacterial growth while inhibiting harmful bacteria, contributing to overall gut health and enhanced nutrient digestion.

The C60 group bonded to at least one of vanadium, bismuth, or gallium metal cation M, 670, allows the delivery of bonded pyruvate, 680, and bonded citrate having at least one available hydroxyl group 620, 640 across the lipid barrier of the cell and organelle membranes such as the cristae of mitochondria, to perform the following functions. The citrate and pyruvate groups directly reduce and eliminate reactive oxygen species, particularly hydrogen peroxide (H2O2). Both the bonded citrate and any citric acid metabolized and released from the nanoparticle compound enhances the expression of heat shock protein genes such as HSP70 in mitochondria under oxidative stress conditions. HSPs act as molecular chaperones and play a crucial role in protecting cellular proteins from oxidative stress induced damage. The presence of either citric or pyruvate groups introduced by the nanoparticle compound reduces the formation of carbonyl groups in proteins to prevent oxidation, thereby maintaining cellular protein structural and functional integrity. The presence of the citric acid functional group improves the activities of cell enzymes that neutralize reactive oxygen species, such as superoxide dismutase (SOD), catalase (CAT), and peroxidase (POD) by mechanisms that are well understood in the study of cell biology.

The negatively charged nanoparticle compound enables a charge-transfer complex formation, allowing chelation of positively charged, poorly soluble Fe3+ cations from cancer cells. This chelation inhibits phosphofructokinase activation in glycolysis during cancer treatment. The compound's hyaluronic acid group, preferentially catabolized by cancer cells, enhances this ion exchange.

The nanoparticle compound leverages vanadium dioxide (VO₂) and ascorbic acid to enhance chondrogenesis, the formation of cartilage from mesenchymal stem cells. VO₂'s surface, modified by ascorbic acid, creates an interface that influences cell adhesion and differentiation. The metal to insulator phase transition (MIT) in VO₂ is significantly triggered by physiological temperatures and hydrogen atoms from ascorbic acid, however it is more completely generated by exposure to ultrasonic irradiation, which generates localized electrical fields and increases the matrix stiffness, both properties of which are crucial for stem cell differentiation. The ultrasonic irradiation process also helps to create a transient production of reactive oxygen species. The dynamic redox environment also initiates stem cell proliferation. The compound's unique surface chemistry induces chondrogenesis without requiring additional iron supplementation when it is used in the human body because of the presence of surplus iron from blood infused tissues. Ultrasound treatment can be used to trigger break apart calcium phosphate deposits in aged tissues, exposing fresh surfaces for VO₂-bonded ascorbic acid adhesion. This approach offers a controllable platform for directing stem cell fate, combining ascorbic acid's hydrogen donating benefits with VO₂'s unique adhesion and stiffness properties, and the generation of electric fields and currents during MIT, potentially revolutionizing treatments requiring cartilage scaffolding integration, as well as providing damaged or aging connective tissue regeneration and repair.

The nanoparticle compound has applications in various biological tissues, including bone and tooth implants, bone glue, 3D printed organs, and surgically introduced bone sections. It interfaces with endogenous bone and cartilage, treats injuries or necrotic arthritis, and differentiates diverse stem cell types, facilitating healing and tissue reinforcement. The nanoparticle compound's ability to integrate with and reinforce connective tissues extends to the larynx, potentially improving its flexibility and thereby enhancing vocal quality and control.

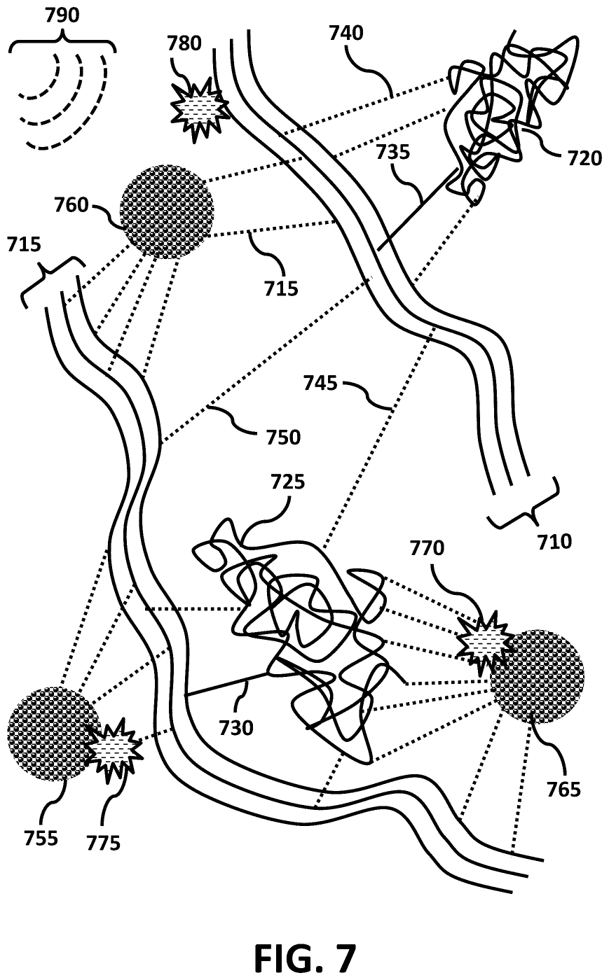

FIG. 7 schematically illustrates nanoparticle compound intercalation of connective tissues along with substance or drug delivery. The figure shows: The nanoparticle compound contains a fullerene group enabling penetration and seepage between multiple layers of tissue, cartilage, and fatty acids, including layered fatty acids of neurons, astrocytes, and nerve structures 710. This allows simultaneous treatment of neurons near inflamed cartilage and proximal inflamed connective tissues, providing holistic pain relief.

Joint and synovial tissue regions between abutting bones contain layered connective tissues comprising: Endogenous collagen fibrils of cartilage 715. Endogenous hyaluronic acid molecules 720, 725. These are bonded together with: At least one covalent bond 730, 735. Multiple hydrogen bonds, indicated by dotted lines 740, 745, 750.

Nanoparticle compound molecular structures 755, 760, 765 can bond with delivered substances, drugs, or stem cells 770, 775, causing tissue reintegration into the matrix of layered tissues, including the cell membranes of nerves 710, and cartilage tissues 715. The nanoparticle compound's ability to form an interpenetrating network with gut tissues strengthens the gut lining, improving barrier function. Additionally, the gallium component of the compound promotes ionic calcium deposition in bone tissues, helping to mitigate osteoporosis.

Substance release is shown by 760, 780. This release can be accelerated in a directed manner by applying ultrasonic irradiation energy, indicated by three dashed arcs in the bracketed region 790. Ultrasound irradiation 790 activates stem cells proximal to the nanoparticle compound 780, accelerating their differentiation to provide healing and tissue reinforcement. Other deliverable substances may include analgesics such as aspirin (salicylic acid) or cannabidiol. The commercially available product Pepto-Bismol's active ingredient is bismuth subsalicylate, which has the chemical formula C₇H₅BiO₄S. This compound consists of bismuth (Bi), salicylic acid (C₇H₆O₃) components, and an additional bridging oxygen atom. The provided metal cations gallium, vanadium, and bismuth are compatible with this raw material and will incorporate it into any of the coordination complexes and bonded structures among the tissues shown. Other analgesic substances that have at least one hydroxyl group, such as cannabidiol, are likewise able to form complexes or reversibly bridging oxygen from such hydroxyl functional groups when metabolized at physiological conditions; this enables effective drug transport by the nanoparticle compound. The nanoparticle compound and optional drug delivery by it is able to provide neuroprotection, crucial when inflammation is accompanied by pain, as the nerves in or near inflamed tissues require simultaneous treatment.

The slow release of pyruvic and citric acids from the nanoparticle compound positively impacts gut health by maintaining the gastrointestinal tract pH, wherein the pyruvic acid particularly favors the growth of beneficial probiotic lactic acid bacteria (LAB). This process may be part of a pH dependent bacterial quorum sensing communication process. The presence of hyaluronic acid in the nanoparticle compound reinforces the intestinal mucus layer as a crucial mechanism in support of gut health. Citric, pyruvic, and hyaluronic acids can enhance gut barrier function by forming complexes with and therefore modulating the tight junction proteins. The nanoparticle compound can also alter gut microflora demographics by enhancing the availability of specific amino acids during nutrient digestion. This is accomplished by forming pi-pi interactions on the carbon-based C60 functional group, which plays a crucial role in the adsorption of the nanoparticle compound to enhance the digestion and delivery of three essential amino acids. The decreasing curvature of the nanoparticle compound on dispersion and their metabolization leads to increased intestinal adsorption rates of these essential amino acids. Tryptophan (Trp) contains an indole ring, which is likely to form strong pi-pi interactions with C60; phenylalanine (Phe) has a benzene ring, which can participate in pi-pi interactions with C60; histidine (His) contains an imidazole ring, which may form pi-pi interactions, though weaker than Trp and Phe. These three essential amino acids are also the ones that can be the most subject to dietary insufficiency, especially in aged individuals or in those with gut dysbiosis.

The restoration of homeostasis in damaged or diseased tissues involves the critical roles of stem cell division and differentiation. Adult stem cells are essential for maintaining tissue integrity and facilitating repair by continuously generating differentiated progeny to replace damaged or lost cells. This process is regulated by complex signaling networks, such as the hedgehog (Hh) pathway, which involves transcriptional factors like Gli1 in mesenchymal stem cells. These cells respond to injury by proliferating and differentiating into the necessary cell types to restore tissue function. The nanoparticle compound's ability to stimulate stem cell division and differentiation, coupled with its anti-inflammatory and tissue-regenerative properties, accelerates the restoration of homeostasis in damaged or diseased tissues.

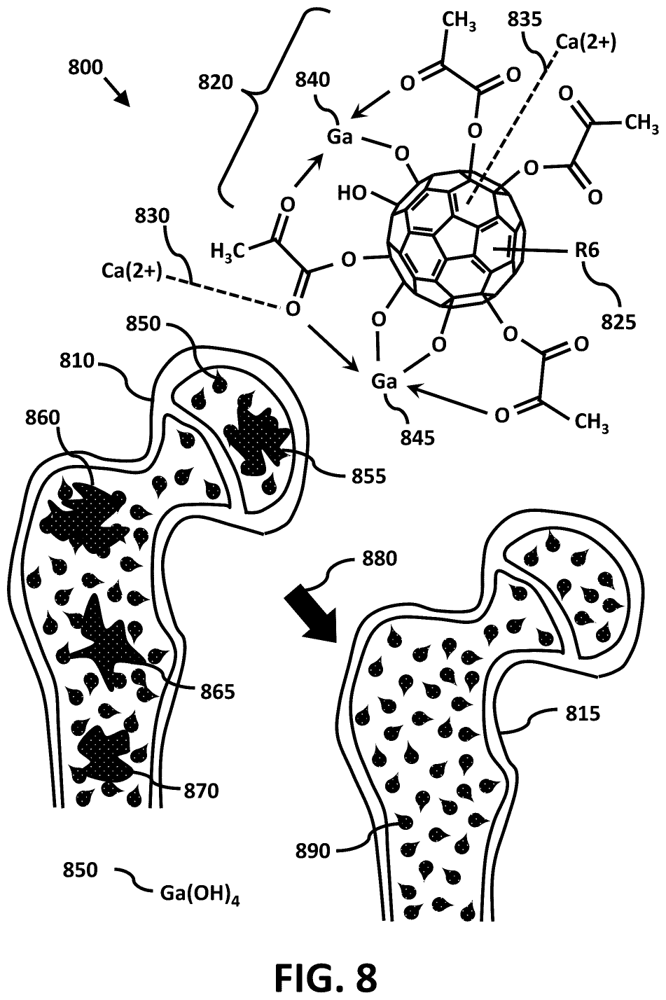

FIG. 8 illustrates nanoparticle compound-enhanced bone density recovery for osteoporosis. The figure shows: Natural or 3D-printed bone scaffold inserted into bone structures may lack dense bone regions, 810. Nanoparticle compound introduction aims to densify this bone, 815. A portion of the nanoparticle compound structure is shown, 820. Other parts of the nanoparticle compound are denoted by the functional group R6, 825 (e.g., hyaluronic esters, citric esters, and metal oxide bridges). The nanoparticle compound forms multiple pi complexes, indicated by dashed lines between calcium and carbonyl groups, 830; and dashed lines with the carbon face of the C60 group, 835. Calcium-bonded nanoparticle compound attracts osteoclasts, promoting calcium retention and aiding in biological bone densification. Nanoparticle compound gallium cations 840, 845 raise pH by gallium donation as Ga(OH)4, 850. Osteoporotic bones exhibit enlarged cavities 855, 860, 865, 870. Nanoparticle compound, administered via diet or surgical implant, bonds with calcium in bone 810, promoting gallium oxidation 840, 845, thereby attracting osteoclasts for bone regrowth and densification, 880. The resultant densified bone has smaller cavities 895 with new hydroxyapatite crystallization. The administered nanoparticle compound can integrate with native bones or printed tissue scaffolds; accelerate bone recovery and integration via like-type bonding and reinforcement; and significantly remediate osteoporosis.

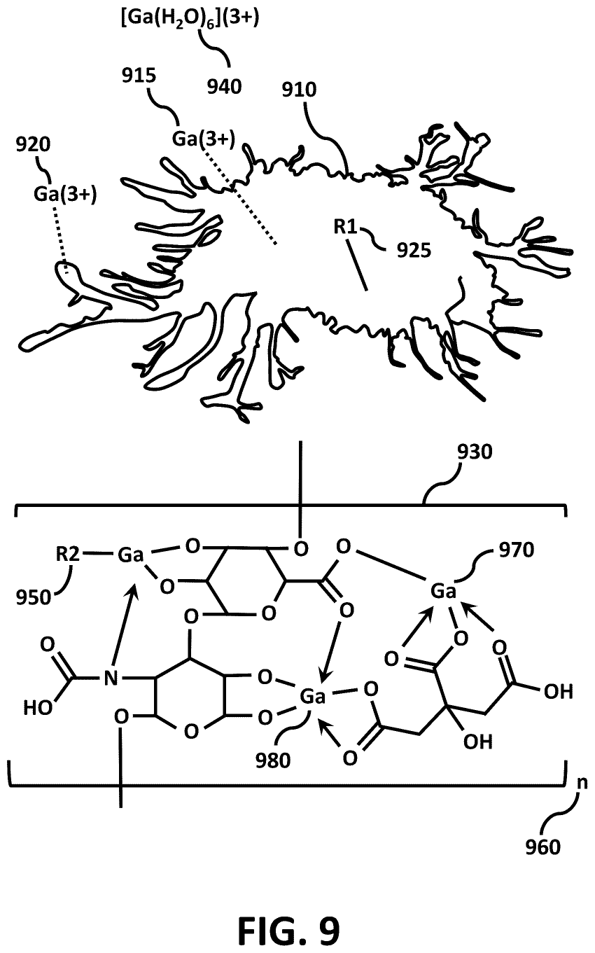

FIG. 9 illustrates the use of nanoparticle compound to treat hypoxic cancer cells and neoplasms. Proliferative neoplasm 910, a common cell type in tumors and cancers, attracts and binds solvated gallium ions 915 and 920, inducing apoptosis. Nanoparticle compound incorporates esterified gallium and citrate with anti-cancer properties, synergistically combating cancer cells at R1, 925. The molecular structure 930 may release hydrated gallium ions over time. R2 represents other structural elements of nanoparticle compound, bonding with R1, 925. The subscript n denotes multiple repeating units within the brackets, nominally ranging from n=4 to 400.

The nanoparticle compound penetrates and becomes part of neoplasms, aided by ambient physiological hyaluronic acid incorporation, forming an interpenetrating network among tumor cells. It can deliver cancer vaccines, chemotherapy, DNA, RNA, and is compatible with radiation therapy to protect healthy cells. The compound inhibits glycolysis, which is a preferred pathway by cancer cells and relies on Fe3+ ion exchange replacement by Ga3+. The antioxidant characteristics of the citric acid group and the C60 group provides free radical quenching to regulate innate immune damage from free radical formation in response to the invasion of cancer cells. The nanoparticle compound counters cancer cell proliferation by reducing glycolysis through the inhibition of phosphofructokinase. The high citrate concentrations delivered by the compound interfere with this key glycolytic enzyme, preferentially affecting cancer cells that rely heavily on glycolysis for energy production.

Gallium is known to be toxic to energy intensive cancer cells while sparing healthy cells with less energy demands operating primary through use of the oxidative phosphorylation (OXPHOS) pathway. The presence of Ga3+ can dilute Fe3+, slowing cancer cell replication without significantly affecting healthy cells. The C60 group of the nanoparticle compound generates a protein corona. Such a protein corona retards the flow of nutrients to cancer cells, which otherwise can grow quickly by the catabolism and destruction of the surrounding proteins extracted from proximal healthy cells and tissues.

The nanoparticle compound acts as a ‘trojan horse,’ delivering gallium through its structure containing pyruvic, hyaluronic, and citric groups. This primarily disrupts glycolysis in fast-growing cancer cells, leaving healthy respiring cells unaffected. It slows the replication of cancer cells by interfering with Fe3+-dependent DNA replication, while allowing for natural replacement of Fe3+ in slower-growing healthy cells through dietary intake.

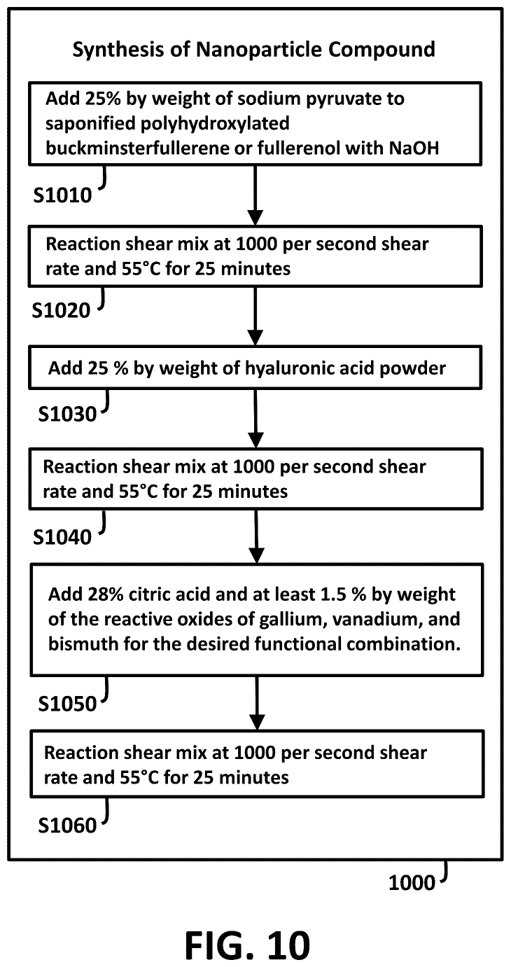

FIG. 10 is a flowchart 1000 illustrating methods of synthesizing the nanoparticle compound of the present invention. In step S1010, 25% by weight of sodium pyruvate is added to commercially available saponified polyhydroxylated buckminsterfullerene (sodium fullerenol). The number of oxygen bridges (oxyl groups) covalently bonded with the buckminsterfullerene group is typically determined by titration. The combined number of hydroxyl and saponified hydroxyl groups may range from 6 to about 24, with a nominal value of about 18. Sufficient sodium hydroxide (NaOH) is added to shift the pH of the dry mixture to greater than 8. Fullerenol may be used as a starting material as it can form the saponified form. Step S1020 involves reaction shear mixing of the combined ingredients at 1000 per second shear rate and 55° C. for 25 minutes. In step S1030, add 25% by weight of hyaluronic acid with a molecular weight of about 4 kilodaltons. The molecular weight may be adjusted to maximize interpenetrating interaction effects with diverse types of connective tissues. Step S1040 comprises reaction shear mixing of the combined ingredients at 1000 per second shear rate and 55° C. for 25 minutes. In step S1050, 28% citric acid and at least 1.5% by weight of combined reactive metal oxides are added to the weight of the intermediate reaction products. The reactive metal comprises enough of each the oxides of gallium, vanadium and bismuth in that combination required to achieve the desired treatment effect. The osteogenic effects of multivalent gallium oxide nanoparticles can begin to exhibit measurable effects at greater than 0.1% by volume which can be adequate for some treatment objectives. The chondrogenic effects of vanadium dioxide nanoparticles of 10 nanometer average diameter can begin to exhibit measurable effects at greater than 1% by volume which can be adequate for some treatment objectives. The spin-wave activation of vanadium dioxide at or above the MIT critical temperature requires at least 20% to 30% of this component by volume to achieve the desired electric fields and currents. This electrical percolation threshold depends on achieving a critical volume fraction where conductive particles can form a continuous network and may require adjustment depending on the tissue matrix or the customized 3D-printed biopolymer scaffolding matrix dielectric properties. The final step S1060 involves reaction shear mixing of the combined ingredients at 1000 per second shear rate and 55° C. for 25 minutes. The metals form metal oxide derivatives bridging some hydroxyl functional groups of the reactive intermediate material to form the final molecular structure of the nanoparticle compound. The final nanoparticle compound product may be solvated in water for combining with a drug or other active substances such as an analgesic, then spray dried to prepare solid forms for administration. The nanoparticle compound can be used to make various products or product forms necessary for administration in accordance with the intent of the present invention.

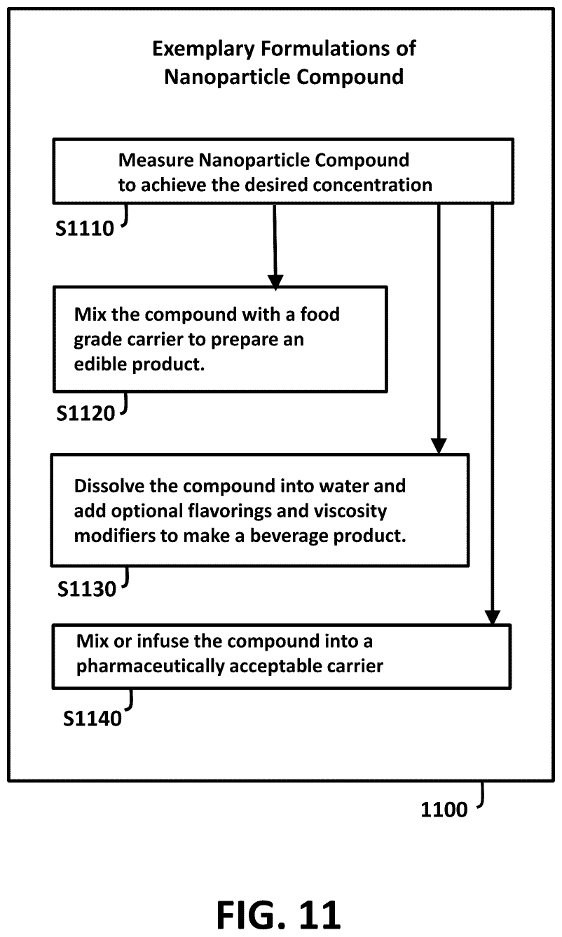

FIG. 11 is a flowchart 1100 illustrating exemplary methods of formulating and administering the nanoparticle compound of the present invention. Step S1110 involves measuring an amount of nanoparticle compound to achieve the desired concentration in a product formulation. In step S1120, the nanoparticle compound is mixed with a food grade carrier such as gelatin, calcium carbonate, or calcium phosphate to prepare an edible or surgically implantable product. This preparation may include the use of common 3D printing technologies to achieve a useful or desirable shape. Common edible products may include gummi bears or powdered additives for reconstitution or mixing with other solid food at a later time. Step S1130 comprises dissolving the nanoparticle compound into water, adding optional viscosity modifiers and preservatives, and adjusting pH as needed to make a dispensable liquid. In step S1140, the nanoparticle compound is mixed or infused into a pharmaceutically acceptable carrier used to make tablets, capsules, or metered paste for personal oral administration. Some of these product forms may comprise infusion into a transdermal patch or a buccal patch designed to dissolve with saliva for ingestion.

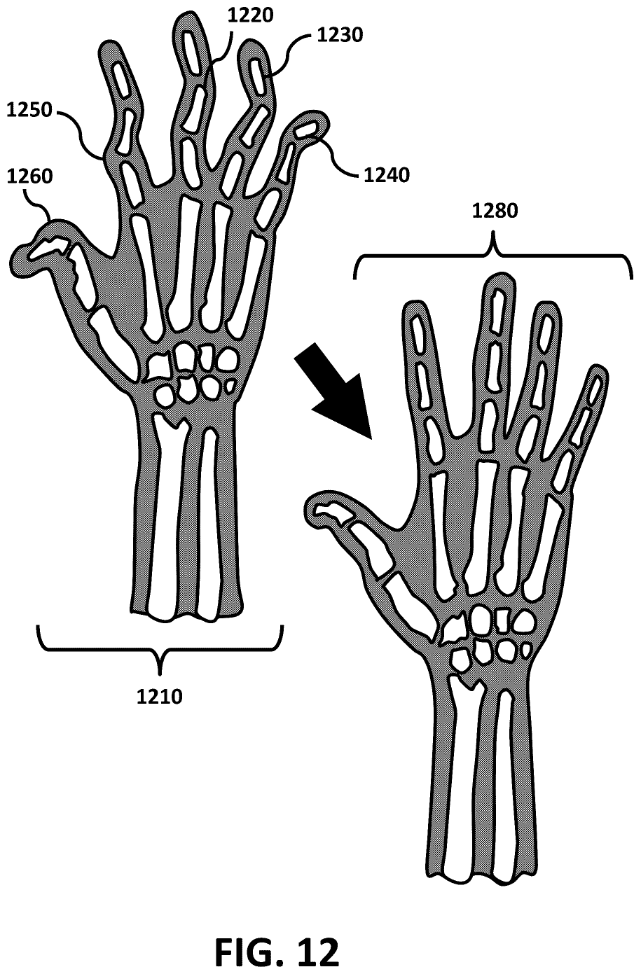

FIG. 12 illustrates the recovery of inflammation and swelling-induced displacement of joints on cartilage remodeling after treatment with nanoparticle compound. The illustration represents a human hand, exemplifying effects applicable to other body parts with cartilage and synovial tissues, such as the spine or knee. The cross-section of a user or patient hand 1210 shows displacement of finger bones 1220, 1230, 1240, visible externally as extended swellings 1250, 1260, typical of arthritis. These displacements may be treated by chiropractors, including vertebral bone adjustments, encompassing all skeletal bone displacements. The nanoparticle compound treatment aims to reverse common forms of arthritis by addressing root causes, resulting in limb configuration restoration and swelling resolution, as illustrated by the treated hand 1280.

Gallium-containing layered double hydroxide coatings have shown promise in enhancing osteogenic cell differentiation and bone mineralization near prosthetic implants. The present invention improves upon this by using nanoparticle compound that metabolizes to release gallium, creating an alkaline environment favorable for calcium deposition and reducing bone porosity. Ultrasonic energy application enhances this gallium delivery, while vanadium oxides in the compound generate electrical energy to stimulate stem cell differentiation. This approach provides customized medical dosage prescription and targeting for the stimulation of osteogenic cell activity in specific bones and teeth.

For common long term maintenance and oral supplement practices, 5 drops of a 500 ppm solution of the nanoparticle compound are adequate per person per day and is equivalent to approximately 0.00179 mg per kg of body weight, assuming an average adult body weight of 70 kg. The actual body weight can vary significantly between individuals, and the amount of tissue damage requiring reinforcement and regeneration can vary, which would affect the final mg per kg value in a recommended dose. For arthritis treatment, the dosage of nanoparticle compound may be adjusted to 0.1 mg/Kg to 5 mg/Kg, depending on the severity of the condition and the specific type of arthritis being treated. The treatable arthritis types include:
Osteoarthritis (OA): The nanoparticle compound forms an interpenetrating network to coat, scaffold, reinforce, and reverse joint cartilage breakdown on a molecular scale.
Rheumatoid Arthritis (RA): The compound intercalates the synovium, repairing degradation due to various factors triggering abnormal immune responses.
Psoriatic Arthritis (PsA): The nanoparticle compound incorporates into affected tissues, providing antimicrobial protection, anti-inflammatory effects, and tissue cohesion restoration through ionomer-mediated electrostatic bonding reinforcement.
Ankylosing Spondylitis (AS): The compound serves as an anti-inflammatory material, restoring compressive modulus and functional deformability to cartilage in spine and sacroiliac joints.
Juvenile Idiopathic Arthritis (JIA): Treatment restores compressive modulus and functional deformability to cartilage and synovial tissues in growing children.
Systemic Lupus Erythematosus (SLE): The nanoparticle compound addresses joint pain, swelling, and damage caused by this autoimmune disease.
Osteonecrosis: The compound restores blood flow through microcapillary repair, allows osteoclastic stem cells to retain calcium phosphate, and provides adhesion restoration and reinforcement of cartilage to regenerated bone tissues.

The nanoparticle compound provides a multifunctional treatment with neural repair properties, usable in combination with other treatments. However, consultation with healthcare professionals is advised for accurate diagnosis and appropriate medical management of bone displacement, chronic pain, inflammation, and arthritis.

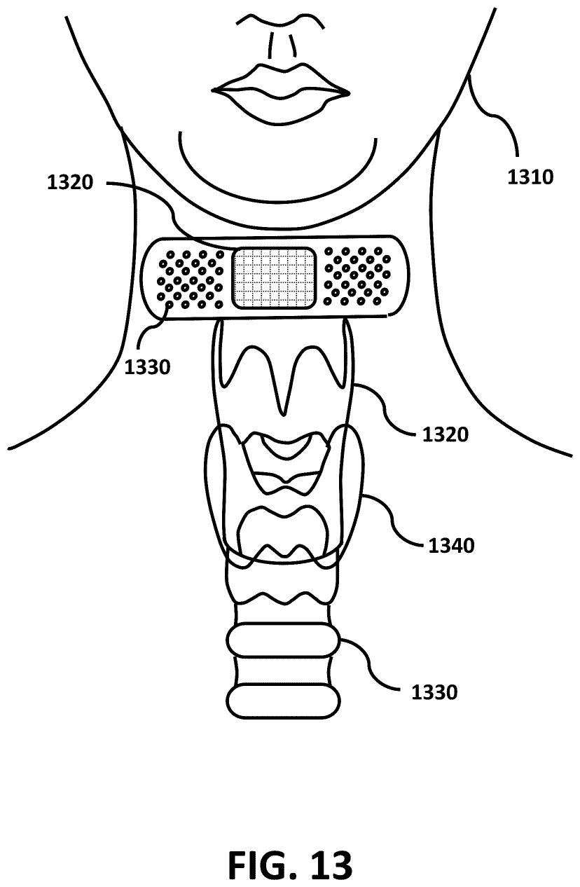

FIG. 13 illustrates treatment to recover larynx cartilage flexibility after tissue remodeling. A user or patient may apply a transdermal patch 1320 provided with a traditional chitosan absorbent pad infused with the nanoparticle compound of the present invention to provide a topical infusion treatment. The transdermal patch is secured to the skin by a temporary adhesive with a flexible substrate flange 1330. Treatment may also include oral supplements of nanoparticle compound to improve vocal dexterity. The combined use of the transdermal patch at night and oral solutions during the day can provide complementary delivery methods of the nanoparticle compound to accelerate the healing process.

Voice disorders can result from changes in laryngeal tissues, and voice quality is closely related to the structure and function of the larynx. Biopolymers have been extensively utilized in tissue engineering such as in creating 3D printed organ scaffolds seeded with stem cells, due to their high biocompatibility, non-immunogenicity, and low cytotoxicity. This provides the context for the addition of nanoparticle compound by ultrasonic assisted incorporation into the original tissue or when added to surgically implanted scaffolded tissues in laryngeal tissue engineering. Voice disorders can result from changes in laryngeal tissues, and voice quality is closely related to the structure and function of the larynx. The application and incorporation of the nanoparticle compound as an interpenetrating biopolymer network infusion for the larynx is to provide structural support to help reinforce or reshape damaged or weakened laryngeal tissues, potentially improving vocal fold closure and vibration, to promote cell growth and tissue regeneration in the larynx, and to repair damage from surgery or disease. The nanoparticle compound network is designed to release anti-inflammatory bismuth containing salicylates or other beneficial substances over time, supporting healing and reducing complications. By carefully selecting biopolymers, the nanoparticle compound infusion can optimize the viscoelastic properties of vocal fold tissues, enhancing their vibration characteristics. The composition of the biopolymer network is to be tailored to address specific voice disorders or patient needs. Compared to more invasive surgical procedures, a biopolymer infusion of nanoparticle compound followed by ultrasonic activation of stem cells will offer a less invasive option for voice quality enhancement by providing precise timing and targeted energy delivery methods directed to specific laryngeal structures. Vocal acoustic performance feedback by medical practitioners is expected to be helpful to balance the enhancement of the mechanical properties of the infusion with changes to the natural tissue properties.

The nanoparticle compound molecules diffuse into the larynx tissues 1320, 1330 as well as into the thyroid gland 1340 to generate ionomeric bonds, restoring flexibility and functionality via the formation of interpenetrating nanoparticle compound networks. Potential beneficiaries of this treatment 1310 may include radio and video talk show hosts, singers, and speech makers who rely on having a strong voice and may experience strain and wear through extensive vocal use. In cases of severe damage, it may become medically necessary to rebuild parts of the larynx, such as by inserting three-dimensional printed medical scaffolding to remodel or replace damaged tissues. The nanoparticle compound is introduced to reinforce such three-dimensionally printed hybrid tissues, which have a matched or compatible shape and composition to the remaining native parts of the larynx. These tissues can include seeded replacement cells and substances that may benefit from interpenetration with nanoparticle compound for integration with living tissues. The use of nanoparticle compound formulations is anticipated to relieve discomfort, alleviate scratchy or raspy voices, and potentially extend the quality and career lifetime of vocalists.

More generally, the nanoparticle compound promotes integration with various types of 3D-printed tissues by forming an interpenetrating network with the scaffold material, enhancing cell adhesion and promoting the growth of seeded cells. The printing process may be performed by adding the nanoparticle compound to other necessary supporting biopolymers, for example a sodium alginate that is compatible with the nanoparticle compound, in locations to support the advanced design, placement, and orientation of the seeded stem cells.

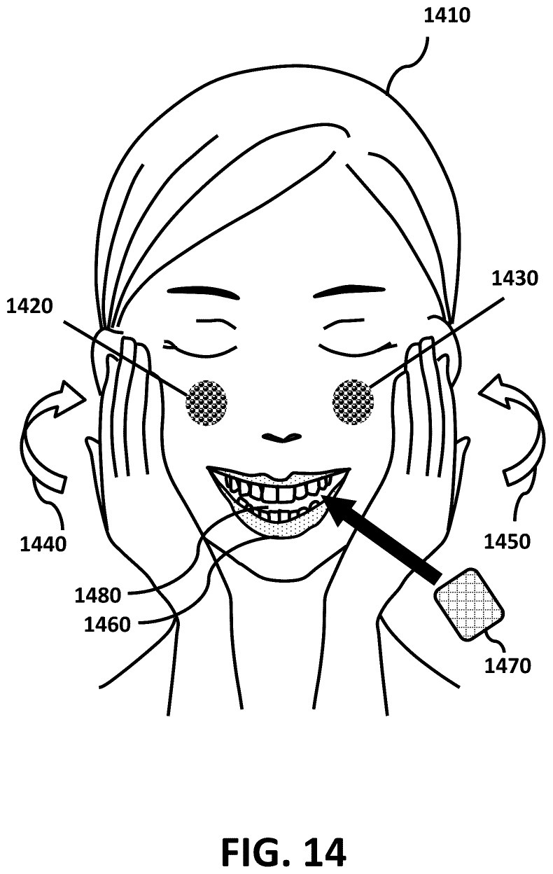

FIG. 14 illustrates the topical and buccal use of the nanoparticle compound of the present invention. For healing lacerations, chapped lips, burns, and sunburns, a user 1410 may apply nanoparticle compound by topical skin administration. The compound can be administered in various forms, including semi-liquid slurry dispersion, cream, ointment, or lotion. Customized formulations may include scents for topical skin use. The skin-care formulation containing nanoparticle compound confers topical antimicrobial, anti-aging, and skin brightening functions, and promotes resistance to the onset of skin cancers. Hyaluronic acid is commonly used to adjust viscosity. The skin care formula can be applied to accelerate the healing of skin surface lacerations, or to promote healing from damage by abrasion, windburn, or sunburn 1420, 1430. Application can be by means of circular rubbing motions as indicated by the direction of arrows 1440, 1450. For administration to the lips of the oral cavity 1460, a wax may be added to help the nanoparticle compound maintain a coating to moisturize, seal, and accelerate healing of cracked and dried lip surfaces. In the case of buccal administration, a dissolving buccal patch 1470 is placed under the tongue 1480 as indicated by the direction of the large black arrow. In all applications, the nanoparticle compound aids in healing and maintains a protective antibacterial function while promoting tissue elasticity and cohesion.

The nanoparticle compound serves as an effective adjuvant for analgesics such as aspirin or cannabidiol, enhancing their delivery and efficacy through improved tissue penetration and targeted release. By utilizing advanced nanodelivery systems, the nanoparticle compound ensures precise targeting and deep tissue penetration, optimizing drug bioavailability and therapeutic impact. Its controllable surface chemistry facilitates site-specific drug release, increasing the concentration of the analgesic at the target site while minimizing systemic exposure. Additionally, the use of responsive biomaterials within the nanoparticle compound prolongs drug action and enhances precision therapy, particularly in dense tissue environments that typically impede drug absorption. Techniques such as ultrasound-induced cavitation can further augment these effects by promoting deeper penetration and controlled release within target tissues. This integrated approach significantly improves therapeutic outcomes, making the nanoparticle compound a promising adjuvant for enhancing analgesic efficacy.

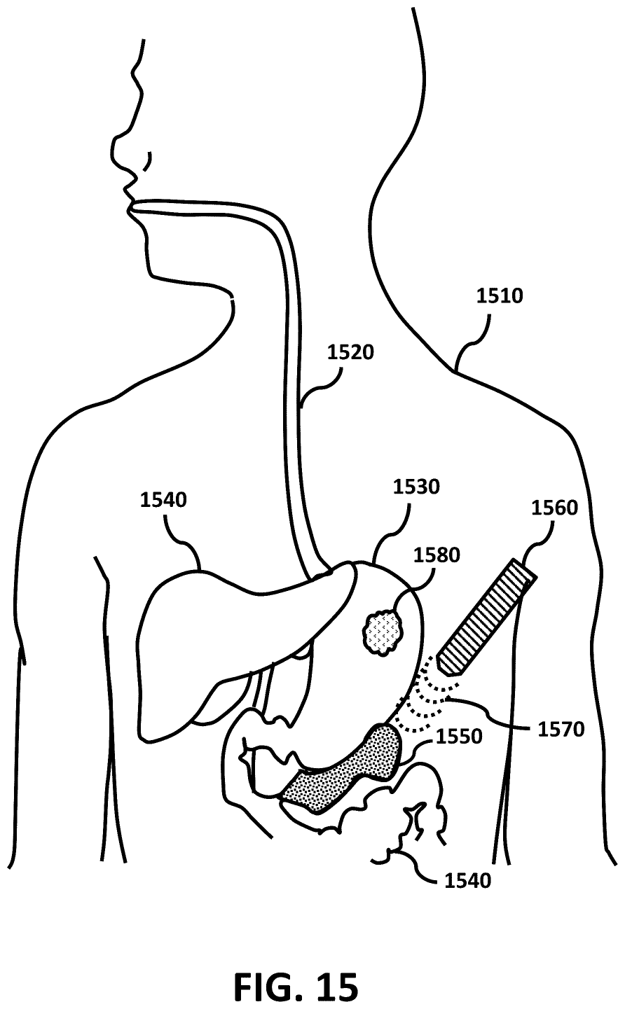

FIG. 15 illustrates the non-contact activation of the nanoparticle compound inside a targeted internal organ by external application of ultrasonic energy. A patient 1510 may ingest the nanoparticle compound through the esophagus 1520, passing through the stomach 1530 positioned near the liver 1540. Any organ may be targeted for nanoparticle compound activation by directed irradiation; the pancreas 1550 is selected as a non-limiting example. Alternative methods of nanoparticle compound introduction include direct injection, endoscopic device delivery, or implantation of sutures or surgical threads containing the compound.

An ultrasound generating device 1560 directs ultrasonic energy from outside the human body 1510 to the desired region of the targeted organ, represented by dotted arcs 1570. This irradiation may be supplied externally or via an endoscopic or enteric device. The ultrasonic energy induces electric voltages through spin-wave oscillations in the vanadium II oxide component of the nanoparticle compound, conducted through the fullerene carbon nanoparticles. This activation can stimulate stem cell growth, for example, to replace dysfunctional pancreas cells in diseases such as diabetes mellitus type 2. The nanoparticle compound may also be deposited into discontinuous regions such as a stomach ulcer 1580. It integrates with and enhances the barrier properties of mucus, the protective biopolymer lining wet epithelia. Ultrasonic activation generates biochemical bonding and crosslinking between the nanoparticle compound and endogenous mucus. The nanoparticle compound provides a molecular scaffold that bonds to and integrates with stomach mucus, optionally carrying medicinal drugs or fluorescent indicator dyes for diagnostic and therapeutic purposes. This molecular reinforcement bridges gaps in stomach ulcers 1580 or treats conditions like inflammatory colitis in the intestine 1540. This method enables non-contact induction of electrical voltage oscillations and electrostatic charge accumulation within living tissue to accelerate healing and stimulate stem cell growth and division, without the need for wires to introduce electrical stimulation.

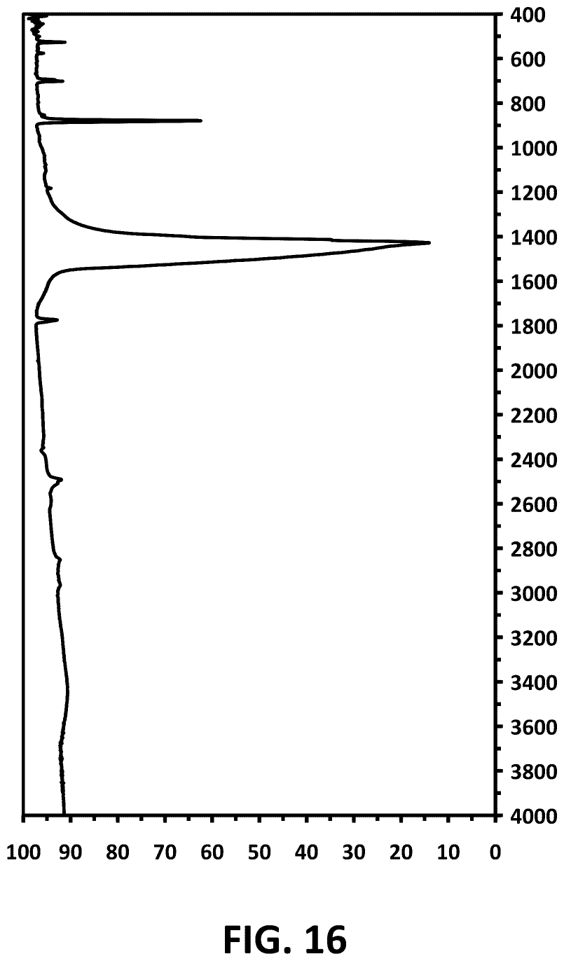

FIG. 16 illustrates experimental FTIR data for the sodium salt of polyhydroxylated buckminsterfullerene, a raw material for nanoparticle compound synthesis. Sample preparation for this and subsequent FTIR analyses follows the ‘KBr pellet’ method. This method involves mixing, crushing, and consolidating under 7 metric tons of pressure approximately 0.001 grams of analyte with 1 gram of anhydrous potassium bromide (KBr). This process forms a translucent pellet of about 0.4 mm thickness. Spectral background subtraction is performed using a control pellet of pure KBr. The Fourier transform infrared spectrophotometer used is a model RF6000 FTIR instrument manufactured by Shimadzu of Japan. The FTIR spectrum of the sample reveals several key features. A weak, broad band at 3450 cm⁻¹ is attributed to the hydroxyl functional group. A narrow, strong absorbance peak at 1428 cm⁻¹ arises from carbon-carbon bonds of the buckminsterfullerene group. A sharp absorbance band at 880 cm⁻¹ is attributed to Fullerenol carbon-oxygen stretching (C—O), reported in literature as a (C—ONa) vibration for dry alkaline treatment of fullerene with sodium hydroxide. Notably, there is an absence of carbon-hydroxyl (C—OH) absorption in the 1370 cm⁻¹ region, presumably due to sodium substitution for hydrogen. Characteristic delocalized carbon-carbon absorbances of the fullerene carbon structure appear at 576 cm⁻¹ and 526 cm⁻¹. The saponified Fullerenol, identified by FTIR as polyhydroxylated Fullerenol treated with sodium hydroxide, is to be used in synthesizing the nanoparticle compound of the present invention. It is noted that chemical esterification reactions proceed best under dry solid conditions away from neutral pH, such as at alkaline or acidic conditions.

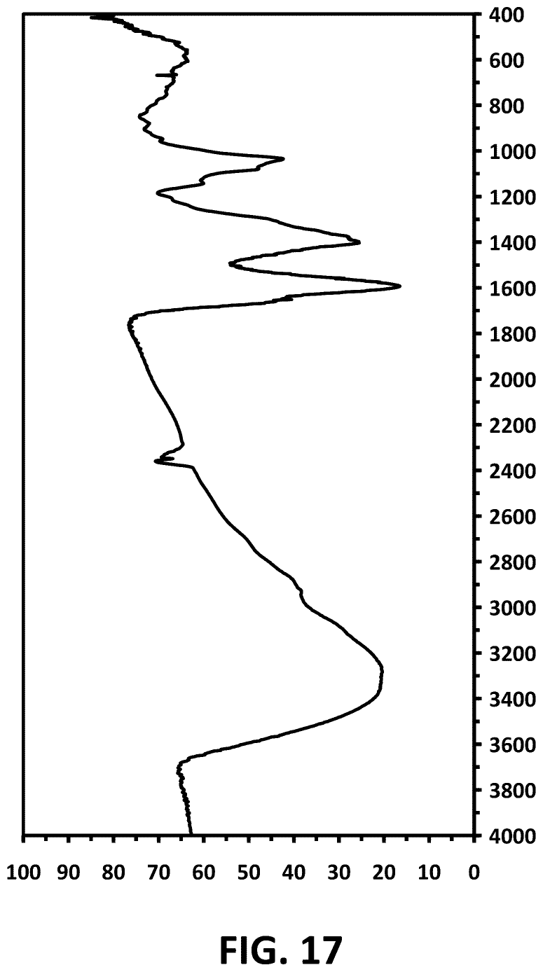

FIG. 17 illustrates experimental FTIR data for hyaluronic acid (HA) raw material that can be used for the nanoparticle compound synthesis. The FTIR spectrum reveals several significant absorption bands characteristic of hyaluronic acid. A strong absorption band is observed at 3325 cm⁻¹, indicating the presence of both hydroxyl (OH) and amine hydrogen (NH) stretching vibrations. The negatively charged deprotonated acid carboxylate functional group (COO—) manifests in two distinct absorbances. The band at 1590 cm⁻¹ is attributed to the antisymmetric stretching vibration, while the absorbance at 1403 cm⁻¹ corresponds to the symmetric stretching vibration of this group. Additionally, the spectrum exhibits absorbances at 1121 cm⁻¹, 1069 cm⁻¹, and 1035 cm⁻¹, which are attributed to carbohydrate contributions in the hyaluronic acid structure. These spectral features confirm the identity and purity of the hyaluronic acid raw material, validating its suitability for use in the nanoparticle compound synthesis of the present invention.

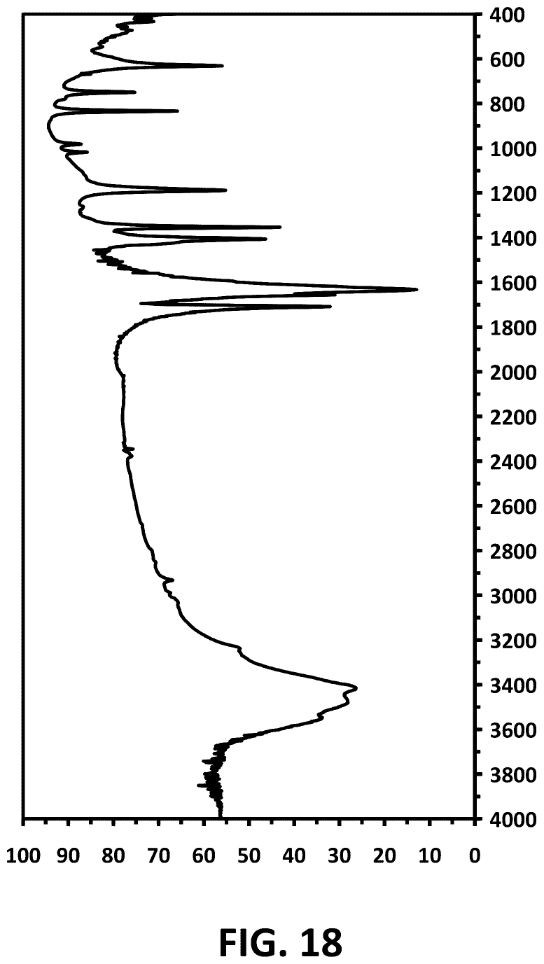

FIG. 18 illustrates experimental FTIR data for sodium pyruvate raw material that can be used for the nanoparticle compound synthesis. The FTIR spectrum of sodium pyruvate exhibits several characteristic absorption bands. A strong band is observed at 1633 cm⁻¹, which is attributed to the anti-symmetric stretching frequency of the carboxylate group. Correspondingly, a medium intensity band located at 1406 cm⁻¹ represents the symmetric stretching frequency of the same carboxylate group. A distinct band centered at 1709 cm⁻¹ is attributed to the stretching frequency of the carbonyl ketone (C═O) group in the pyruvate molecule. This band is characteristic of the ketone functional group present in pyruvic acid and its salts. Two additional bands provide information about carbon-oxygen (C—O) bonds in different chemical environments within the sodium pyruvate molecule. The band at 1353 cm⁻¹ is attributed to the C—O stretching vibration of the ester group bonded to sodium. In contrast, the band at 1188 cm⁻¹ corresponds to the C—O stretching vibration of the bond attached to the carbonyl carbon. These spectral features collectively confirm the identity and purity of the sodium pyruvate raw material, validating its suitability for use in the nanoparticle compound synthesis of the present invention.

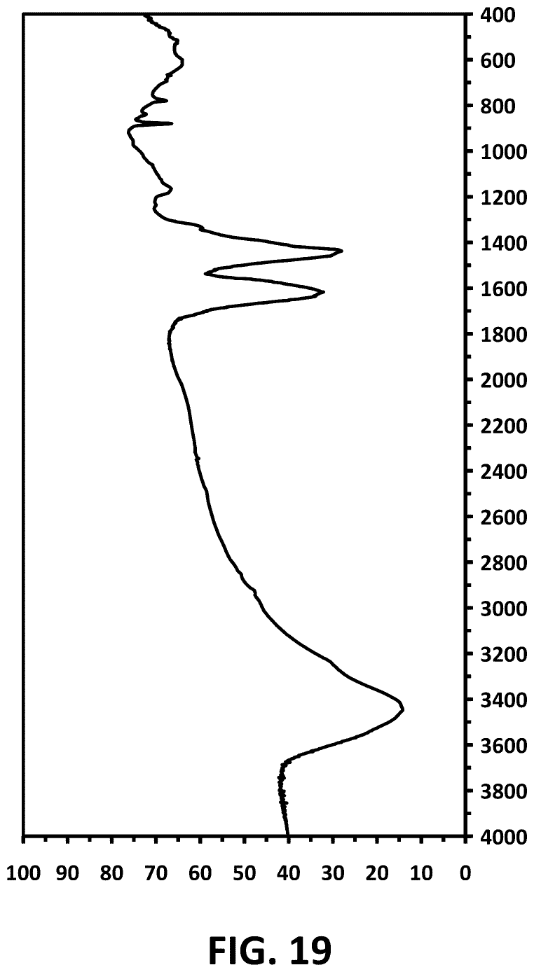

FIG. 19 illustrates experimental FTIR data for the pyruvate ester of buckminsterfullerene, a reactive intermediate material formed during one part of the nanoparticle compound synthesis process. The FTIR spectrum reveals significant changes in absorption bands compared to the starting materials, indicating successful formation of the buckminsterfullerene pyruvate ester. A notable shift is observed in the absorption band previously associated with saponified fullerenol. The band that appeared at 1428 cm⁻¹ in FIG. 14 has now shifted significantly to 1438 cm⁻¹. This shift provides strong evidence for the successful formation of the buckminsterfullerene pyruvate ester bond (C60-O—C). Furthermore, the spectrum shows a shift in the carbonyl (C═O) bond absorption. The band that was originally observed at 1633 cm⁻¹ in the starting pyruvate material now appears at 1617 cm⁻¹. This shift is indicative of the formation of a pi-carbonyl bonding interaction with the delocalized carbons of the C60 group. These spectral changes, particularly the shifts in absorption bands, provide strong evidence for the successful synthesis of the pyruvate ester of buckminsterfullerene. This intermediate material represents a crucial step in the overall nanoparticle compound synthesis process of the present invention.

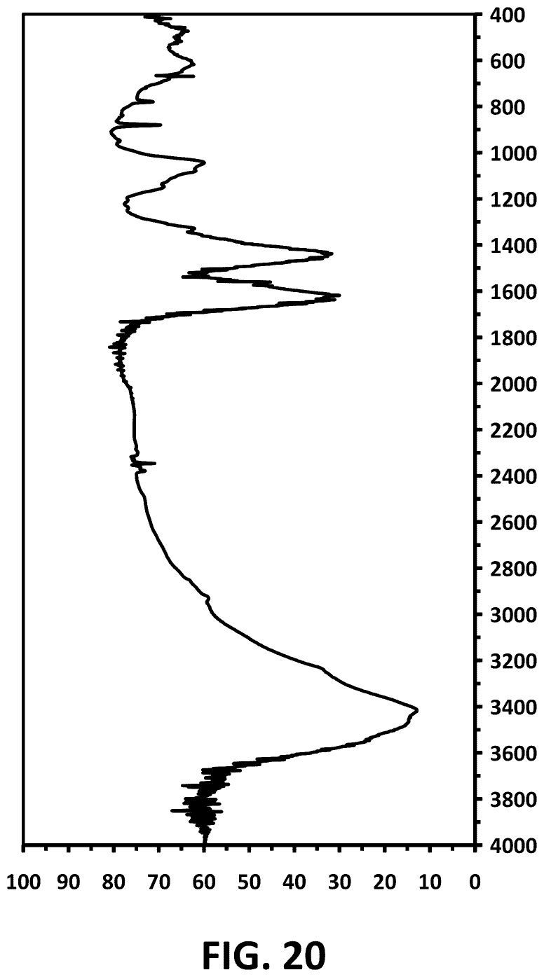

FIG. 20 illustrates experimental FTIR data for the hyaluronic pyruvate esters of buckminsterfullerene, a reactive intermediate material formed in another part of the nanoparticle compound synthesis process, both with and without the presence of gallium oxide. The FTIR spectrum reveals several key features indicating the preservation of certain molecular structures and the formation of new bonds. The pyruvate absorbance at 1617 cm⁻¹ remains unchanged, suggesting that the pi-carbonyl bonding interaction with the delocalized carbons of the C60 fullerene group is preserved from the fullerene pyruvate ester starting material. Similarly, the asymmetric stretching at 1438 cm⁻¹ remains constant, indicating that the fullerene pyruvate ester (C60-O—C) bond remains intact. Significant shifts are observed in the carbohydrate absorbances originally associated with pure hyaluronic acid; the bands previously observed at 1121 cm⁻¹, 1069 cm⁻¹, and 1035 cm⁻¹ now appear at 1147 cm⁻¹, 1075 cm⁻¹, and 1044 cm⁻¹, respectively. While the peak shapes remain unchanged, the shift in absorbance wavenumbers indicates the new chemical environment resulting from ester bonding with the saponified fullerenol. Notably, the addition of 5 percent by weight gallium to the reaction mixture does not produce any observable changes in the FTIR spectrum. This suggests either that there are no measurable absorbances for the gallium metal oxide when it interacts with and interposes within the ester bonds as (O—Ga—O), or that any metal oxide absorbance is indistinguishable from that of a carbon ester (C—O) when comprising the structure of gallium hyaluronic pyruvate ester. These spectral features collectively provide evidence for the successful formation of hyaluronic pyruvate esters of buckminsterfullerene and offer insights into the molecular structure of this reactive intermediate in the nanoparticle compound synthesis process.

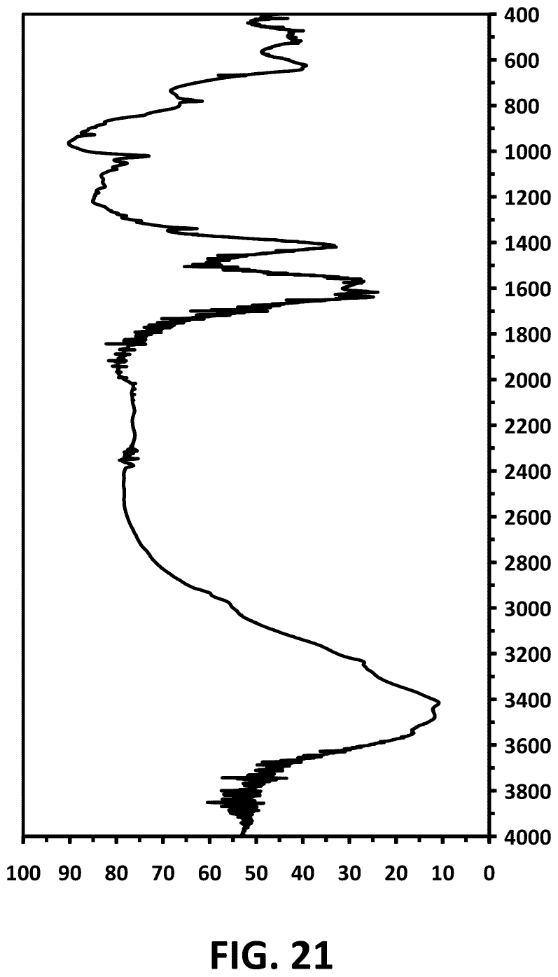

FIG. 21 illustrates experimental FTIR data for the gallium metal oxide citric hyaluronic pyruvate ester of buckminsterfullerene, representing the final nanoparticle compound substance of the present invention. The FTIR spectrum reveals significant changes in absorption bands compared to the intermediate materials. Carbonyl absorbances at 1618 cm⁻¹, 1617 cm⁻¹, and 1570 cm⁻¹ show increased intensity, attributable to the addition of carboxylic acid functional groups to the molecular structure of the conjoined esters. A notable shift is observed in the overall ester absorbance, moving from 1438 cm⁻¹ to 1413 cm⁻¹. This shift indicates that the pyruvate components within this structure have reacted with the citric components, resulting in the formation of a hybrid molecular ester. The addition of gallium, bismuth, vanadium, or their oxides to this structure does not produce any alteration in the measured FTIR absorptions. This suggests that the metal atom bridges between the existing oxygen-bridged components do not affect the vibrational characteristics of the bridging oxygen atoms. The absorbance data presented in this FTIR spectrum is considered characteristic of the nanoparticle compound of the present invention. These spectral features collectively provide evidence for the successful synthesis of the final nanoparticle compound and offer insights into its molecular structure.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A nanoparticle compound comprising:
a buckminsterfullerene C60 bonded to ester functional groups of pyruvic acid, citric acid, and hyaluronic acid; and
oxygen-bridged components selected from the group consisting of vanadium, gallium, and bismuth; and
wherein at least one component is configured to enable quantum-mechanical spin-wave activation.

2. A method of activating and delivering the nanoparticle compound of claim 1, the method comprising:
applying on-contact ultrasonic irradiation to activate the nanoparticle compound composition;
inducing electrical induction within the nanoparticle compound; and
modulating the delivery rate of the nanoparticle compound through directed ultrasound wherein the electrical induction is facilitated by quantum-mechanical spin-waves.

3. A method of treating a subject with the nanoparticle compound of claim 1, the method comprising:
administering the nanoparticle compound to treat arthritis via at least one administration method selected from the group consisting of topical, oral, injection, implantation, and infusion;
wherein the nanoparticle compound forms an interpenetrating network with native tissues; and
enhancing tissue integration and accelerating healing in the subject; and
providing neuroprotection and anti-inflammatory effects to the subject.

4. The nanoparticle compound of claim 1, formulated as a food supplement for enhancing gut health, wherein:
the food supplement controls gut inflammation and dysbiosis, alters gut microflora, and enhances nutrient digestion;
the gallium component exhibits antibacterial activity against gut pathogens and promotes beneficial bacterial growth; and
the compound alters gut microflora demographics by enhancing availability of specific amino acids.

5. The nanoparticle compound of claim 1, wherein the composition is formulated as a food supplement for improving barrier function, the food supplement being effective to reinforce gut lining and resolve conditions associated with intestinal inflammation.

6. The nanoparticle compound of claim 1, wherein the composition is formulated as an anti-inflammatory food supplement effective to provide anti-inflammatory properties and reinforce connective tissue.

7. The nanoparticle compound of claim 1, wherein the gallium component creates an alkaline environment favorable for ionic calcium deposition to promote osteogenic cell differentiation, and the vanadium component simultaneously enhances chondrogenic cell differentiation.

8. The nanoparticle compound of claim 1, wherein the composition is formulated as a food supplement for enhancing voice quality, the food supplement being effective to improve larynx flexibility and enhance vocal quality and control by forming an interpenetrating biopolymer network infusion in laryngeal tissues.

9. The nanoparticle compound of claim 1, wherein the composition is formulated as an antioxidant food supplement effective to exhibit antioxidant functions wherein the buckminsterfullerene C60 component acts to catalyze free radical recombination and quenching and provides neuroprotection against oxidative stress.

10. The method of claim 3, wherein the administering step comprises treating all forms of arthritis via at least one administration method selected from the group consisting of topical, oral, injection, implantation, and infusion administration in an amount of from about 0.1 mg/Kg to about 5 mg/Kg.

11. The method of claim 3, wherein the enhancing tissue integration and accelerating healing step comprises forming a scaffold for cell adhesion, bridging gaps in living tissues, and incorporating the nanoparticle compound into native tissues.

12. The method of claim 3, further comprising promoting integration of the nanoparticle compound with seeded replacement cells and enhancing compatibility with 3D-printed tissues.

13. The method of claim 3, wherein the providing neuroprotection step comprises addressing neuritis and nervous system inflammation, and wherein the nanoparticle compound exhibits bacteriostatic functions.

14. The method of claim 3, further comprising administering the nanoparticle compound to counter cancer cell proliferation, wherein the gallium component inhibits iron-dependent enzymes in cancer cells, and the citrate component interferes with glycolysis by inhibiting phosphofructokinase.

15. The method of claim 3, further comprising stimulating stem cell division and accelerating restorative homeostasis in the subject through complex signaling networks, such as the hedgehog (Hh) pathway.

16. The method of claim 2, wherein the nanoparticle compound comprises esters of pyruvic, citric, and hyaluronic acids with oxygen-bridged vanadium, gallium, and bismuth, functioning as a biologically compatible ionomer.

17. The method of claim 2, wherein the electrical induction within the nanoparticle compound is provided through electromagnetic voltages induced within a vanadium II oxide component and conducted into and through fullerene carbon nanoparticles of the nanoparticle compound composition.

18. The method of claim 2, further comprising accelerating healing and stimulating stem cell division through electromagnetic oscillations within a vanadium II oxide component of the nanoparticle compound.

19. The method of claim 2, wherein the nanoparticle compound utilizes quantum-mechanical activation of cell growth through quantum spin-waves, enabling electric charge manipulation, wherein the vanadium component enables quantum-mechanical spin-wave activation through its metal-to-insulator phase transition properties.

20. The method of claim 2, further comprising delivering an analgesic selected from the group consisting of aspirin and cannabidiol, wherein the nanoparticle compound serves as an adjuvant for the analgesic by enhancing its delivery and efficacy through improved tissue penetration and targeted release.
